(19) United States
(12) Patent Application Publication
(10) Pub. No.: US 2026/0317396 A1
(43) Pub. Date: Oct. 15, 2026

(54) Fungal Signal Peptides

(71) Applicant: Novozymes A/S, Bagsvaerd (DK)

(72) Inventor: Hiromi AKEBOSHI, Mihama-ku (JP)

(73) Assignee: Novozymes A/S, Bagsvaerd (DK)

(21) Appl. No.: 19/108,396

(22) PCT Filed: Sep. 12, 2023
(86) PCT No.: PCT/EP2023/074982

§ 371 Date: Mar. 3, 2025

(30) Foreign Application Priority Data
Sep. 15, 2022 (EP) 22195778.0
Oct. 19, 2022 (EP) 22202556.1

(51) Int. Cl. C07K 14/76; C12N 9/18; C12N 9/24; C12N 9/30; C12N 15/80

(52) U.S. Cl. CPC C07K 14/76; C12N 9/18; C12N 9/2405; C12N 9/242; C12N 9/2494; C12N 15/80; C12Y 301/01074; C12Y 302/01001; C12Y 302/01039; C12Y 302/01078; C07K 2319/02

(57) ABSTRACT

The present invention relates to nucleic acid constructs comprising a first polynucleotide encoding a signal peptide, e.g., from a fungal glycosidase, and a second polynucleotide encoding an alpha-lactalbumin (ALAB) polypeptide; expression vectors and host cells comprising said nucleic acid constructs; methods for producing ALAB polypeptides; and fusion proteins comprising an ALAB polypeptide and a signal peptide.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to nucleic acid constructs comprising a first polynucleotide encoding a signal peptide, e.g., from a fungal glycosidase, and a second polynucleotide encoding an alpha-lactalbumin (ALAB) polypeptide; expression vectors and host cells comprising said nucleic acid constructs; methods for producing ALAB polypeptides; and fusion proteins comprising an ALAB polypeptide and a signal peptide.

### Description of the Related Art

Product development in industrial biotechnology includes a continuous challenge to increase recombinant protein yields at large scale to reduce costs. Two major approaches have been used for this purpose in the last decades. The first one is based on classical mutagenesis and screening. Here, the specific genetic modification is not predefined, and the main requirement is a screening assay that is sensitive to detect increments in yield. High-throughput screening enables large numbers of mutants to be screened in search for the desired phenotype, i.e., higher recombinant protein yields. The second approach includes numerous strategies ranging from the use of stronger promoters and multi-copy strains to ensure high expression of the gene of interest to the use of codon-optimized gene sequences to aid translation. However, high-level production of a given protein may in turn trigger several bottlenecks in the cellular machinery for secretion of the enzyme of interest into the medium, emphasizing the need for further optimization strategies.

Signal peptides (SPs) are short amino acid sequences present in the amino terminus of many newly synthesized polypeptides that target these into or across cellular membranes, thereby aiding maturation and secretion. The amino acid sequence of the SP influences secretion efficiency and thereby the yield of the polypeptide manufacturing process. Bioinformatic tools such as SignalP and SignalP5 can predict SPs from amino acid sequences, but most cannot distinguish between various types of SPs (Armenteros et al., Nat. Biotechnol. 37:420-423, 2019). Moreover, a large degree of redundancy in the amino acid sequence of SPs makes it difficult to predict the efficiency of any given SP for production of recombinant proteins at industrial scale. There are no tools to predict the efficiency with which a given SP directs the secretion of a target protein of interest (POI) (Owji et al., Eur. J. Cell Biol. 2018, 97, 422-441). In fact, finding an efficient SP to secrete a POI is currently still based on trial and error. It is established that the SP-POI match plays a crucial role in determining secretion efficiency (Peng, C. et al., Front. Bioeng. Biotechnol. 2019, 7, 139) whereas the underlying fundamental parameters remain unknown.

Hence, SP selection is an important step for manufacturing of recombinant proteins, but the optimal combination of signal peptide and mature protein is very context dependent and not easy to predict.

Alpha-lactalbumin (ALAB) is a principal protein of milk. ALAB forms the regulatory subunit of the lactose synthase (LS) heterodimer and beta 1,4-galactosyltransferase (beta4Gal-T1) forms the catalytic component. Together, these proteins enable LS to produce lactose by transferring galactose moieties to glucose. As a monomer, alpha-lactalbumin strongly binds calcium and zinc ions and may possess bactericidal or antitumor activity. A folding variant of alpha-lactalbumin, called HAMLET, likely induces apoptosis in tumor and immature cells.

Recombinant ALAB has the potential to improve the nutritional value in food, beverages, and feed. Recombinant ALAB expression has been reported previously in different organisms including transgenic goats (Yuan Y G et al, J Anal Methods Chem. 2014; 2014:281031. doi: 10.1155/2014/281031). Recombinantly produced phytase is widely used as a feed supplement to effectively improve phosphorous utilization and reduce fecal phosphorous excretion in animals as poultry and pig. Thus, for both polypeptides ALAB and phytase, there is a growing need for access thereto in the respective industries.

Although there are expression systems available, there is a need for increasing yields during recombinant production. One major challenge is the fact that ALAB and phytase both have several disulfide bonds which makes correct expression, folding and secretion challenging. Thus, in order to satisfy the growing demand for recombinant ALAB and phytase it is necessary to provide recombinant expression systems with increased ALAB or phytase yields.

### SUMMARY OF THE INVENTION

The present invention is based on the surprising and inventive finding that expression of difficult-to-express proteins (alpha-lactalbumin and phytase) with a fungal signal peptide provides increased yield when expressed in fungal host cells.

Using the signal peptides of the invention an improved yield of the ALAB product compared to expression of the same ALAB product with other signal peptides was observed, e.g., using the JSP017 SP with SEQ ID NO: 45 the ALAB yield can be increased by 53% relative to JSP 002 (Table 9).

Using the signal peptides of the invention an improved yield of the phytase compared to expression of the same phytase with other signal peptides was observed, i.e., an 2.3-fold increase in phytase expression (Table 4).

Notably, the increased expression was achieved using several, different fermentation protocols at different scales, including microtiter plates (MTP), shake flasks (SF), and laboratory fermentation tanks.

In a first aspect, the present invention relates a nucleic acid construct comprising:
a first polynucleotide encoding a signal peptide having a sequence identity of at least 80% to SEQ ID NO: 45, SEQ ID NO:2, SEQ ID NO:4, SEQ ID NO: 47, or SEQ ID NO: 49; and
a second polynucleotide encoding an alpha-lactalbumin (ALAB) polypeptide having a sequence identity of at least 70% to the polypeptide sequence of SEQ ID NO:6;
wherein the first polynucleotide and the second polynucleotide are operably linked in translational fusion.

In a second aspect, the present invention relates to expression vectors comprising a nucleic acid construct of the first aspect.

In a third aspect, the present invention relates to a fungal host cell comprising in its genome:
a) a nucleic acid construct according to the first aspect; and/or
b) an expression vector according to the second aspect.

In a fourth aspect, the present invention relates to a method of producing a alpha-lactalbumin (ALAB) polypeptide, the method comprising:
a) cultivating a host cell according to the third aspect under conditions conducive for production of the ALAB polypeptide; and optionally
b) recovering the ALAB polypeptide.

In a fifth aspect, the present invention relates to a method of producing a polypeptide having phytase activity, the method comprising:
a) cultivating a host cell according to the third aspect under conditions conducive for production of the polypeptide having phytase activity; and optionally
b) recovering the polypeptide having phytase activity.

In a sixth aspect, the present invention relates to a fusion polypeptide, comprising:
a signal peptide having at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence to SEQ ID NO:45, SEQ ID NO: 2, SEQ ID NO:4, SEQ ID NO: 47, or SEQ ID NO: 49, and
(i) an alpha-lactalbumin polypeptide having at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO: 6, or
(ii) a polypeptide having phytase activity having at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, sequence identity to SEQ ID NO:8.

### SEQUENCE OVERVIEW

SEQ ID NO:1 is the GH26 (JSP 002) signal peptide coding sequence
SEQ ID NO:2 is the GH26 (JSP 002) signal peptide (MFAKLSLLSLLFSSAALG).
SEQ ID NO:3 is the GH16 signal peptide coding sequence.
SEQ ID NO:4 is the GH16 signal peptide (MRLPLVSSTVALLSSASLVAA).
SEQ ID NO:5 is the ALAB coding sequence. (without SP)
SEQ ID NO:6 is the ALAB amino acid sequence. (without SP)
SEQ ID NO:7 is the ALAB coding sequence. (with SP GH26)
SEQ ID NO:8 is the ALAB amino acid sequence (with SP GH26)
SEQ ID NO:9 is the ALAB coding sequence. (with SP GH16)
SEQ ID NO:10 is the ALAB amino acid sequence (with SP GH16)
SEQ ID NO:11 is the phytase coding sequence. (without SP)
SEQ ID NO:12 is the phytase amino acid sequence. (without SP)
SEQ ID NO:13 is the phytase coding sequence. (with SP GH26)
SEQ ID NO:14 is the phytase amino acid sequence (with SP GH26)
SEQ ID NO:15 is the phytase coding sequence. (with SP GH16)
SEQ ID NO:16 is the phytase amino acid sequence (with SP GH16)
SEQ ID NO:17 is the intron between SP GH16/SP GH26 and the ALAB coding sequence
SEQ ID NO:18 coding sequence GH16 SP-intron-ALAB
SEQ ID NO:19 coding sequence GH26 SP-intron-ALAB
SEQ ID NO:20 is the GH26 polypeptide coding sequence from Aspergillus luchuensis
SEQ ID NO:21 is the GH26 polypeptide from A. luchuensis
SEQ ID NO:22 is the GH16 polypeptide coding sequence from Aspergillus luchuensis
SEQ ID NO:23 is the GH16 polypeptide from A. luchuensis
SEQ ID NO:24 is the reference GH13 signal peptide coding sequence
SEQ ID NO:25 is the reference GH13 signal peptide
SEQ ID NO:26 is the reference cutinase (JSP004) signal peptide coding sequence
SEQ ID NO:27 is the reference cutinase (JSP004) signal peptide
SEQ ID NO:28 is the reference GH72 signal peptide coding sequence
SEQ ID NO:29 is the reference GH72 signal peptide
SEQ ID NO:30 is the SP GH16-ALAB expression cassette (promoter-SPGH16-intron-ALAB-terminator)
SEQ ID NO: 31 is the SP GH26-ALAB expression cassette (promoter-SPGH26-intron-ALAB-terminator)
SEQ ID NO:32 is the SP GH72-ALAB expression cassette (promoter-SPGH72-intron-ALAB-terminator)
SEQ ID NO:33 is the SP GH16-phytase expression cassette (promoter-SPGH16-phytase-terminator)
SEQ ID NO: 34 is HA442 primer
SEQ ID NO: 35 is HA451 primer
SEQ ID NO: 36 is HA283 primer
SEQ ID NO: 37 is HA444 primer
SEQ ID NO: 38 is HA450 primer
SEQ ID NO: 39 is HA445 primer
SEQ ID NO: 40 is HA442 primer
SEQ ID NO: 41 is HA233 primer
SEQ ID NO: 42 is HA268 primer
SEQ ID NO: 43 is HA489 primer
SEQ ID NO: 44 is the LYA1_4 (JSP017, Aspergillus luchuensis) signal peptide coding sequence
SEQ ID NO: 45 is the LYA1_4 (JSP017, Aspergillus luchuensis) signal peptide (MKYAAALTAVAALAARAAA)
SEQ ID NO: 46 is the pepesin A (JSP019, Aspergillus niger) signal peptide coding sequence
SEQ ID NO: 47 is the pepesin A (JSP019, Aspergillus niger) signal peptide (MVVFSKTAALVLGLSSAVSA)
SEQ ID NO: 48 is the GH28_9 endo-1,4-alpha-polygalacturonase (JSP008, Aspergillus luchuensis) signal peptide coding sequence
SEQ ID NO: 49 is the GH28_9 endo-1,4-alpha-polygalacturonase (JSP008, Aspergillus luchuensis) signal peptide (MHFLQNAFVAATMGAALAAA)

### Definitions

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

Alpha-lactalbumin: The term “alpha-lactalbumin”, “α-lactalbumin” “α-LA”, “α-LAB”, “ALAB”, or “LALBA” means a polypeptide which forms a functional regulatory subunit of the lactose synthase (LS) heterodimer. ALAB forms the regulatory subunit of the lactose synthase (LS) heterodimer and beta 1,4-galactosyltransferase (beta4Gal-T1) forms the catalytic component. Together, these proteins enable LS to produce lactose by transferring galactose moieties to glucose. Non-limiting examples for ALAB polypeptides are bovine ALAB and human ALAB. ALAB quantification can be carried out as described under the Examples with section “Semi-quantification by MALDI-TOF MS” and “Size exclusion chromatography”.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the polypeptide, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” means any step involved in the production of a polypeptide including, but not limited to, transcription, post-transcriptional modification, translation, post-translational modification, and secretion.

Expression vector: An “expression vector” refers to a linear or circular DNA construct comprising a DNA sequence encoding a polypeptide, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Extension: The term “extension” means an addition of one or more amino acids to the amino and/or carboxyl terminus of a polypeptide, wherein the “extended” polypeptide has phytase activity or wherein the extended polypeptide is an ALAB polypeptide which forms a functional regulatory subunit of the lactose synthase (LS) heterodimer. Persons skilled in the art will know that a polypeptide having a given amino acid sequence and enzymatic activity may be produced with one or a few additional amino acids at the N- and/or C-terminus, and that such a polypeptide can have essentially the same enzyme activity. Such extended polypeptides are intended to be encompassed by the present invention.

Fragment: The term “fragment” as used in the context of a polypeptide means a polypeptide having one or more amino acids absent from its amino and/or carboxyl terminus, wherein the fragment has phytase activity, or wherein the fragment is an ALAB fragment which forms a functional regulatory subunit of the lactose synthase (LS) heterodimer. The fragment may be produced naturally during expression and/or purification of the polypeptide, or may be the result of expression of a modified nucleotide sequence expressing the fragment or of targeted removal of amino acids from the amino and/or carboxy terminus.

Fungal glycosidase: The term “fungal glycosidase” means any glycosidase (EC 3.2.1) that hydrolyses O- and S-glycosyl compounds and which is encoded with an N-terminal signal peptide and natively expressed by a fungal species. The term “fungal glycosidase” also includes, but is not limited to, fungal mannanases (EC 3.2.1.78) and fungal glucanases (EC 3.2.1.39). Other non-limiting examples are alpha-amylase (EC 3.2.1.1), beta-amylase (EC 3.2.1.2) and lysozyme (EC 3.2.1.17).

Fusion polypeptide: The term “fusion polypeptide” is a polypeptide in which one polypeptide is fused at the N-terminus and/or the C-terminus of a polypeptide of the present invention. A fusion polypeptide is produced by fusing a polynucleotide encoding another polypeptide to a polynucleotide of the present invention, or by fusing two or more polynucleotides of the present invention together. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3:568-576; Svetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6:240-248; and Stevens, 2003, Drug Discovery World 4:35-48.

Glucanase: The term “glucanase” means a glucanase comprising a signal peptide at its N-terminal end, the glucanase having endo-b-1,3-glucanase activity (EC 3.2.1.39). A non limiting example for a glucanase is the Aspergillus luchuensis glucanase. The signal peptide sequence derived of the A. luchuensis glucanase is termed “GH16” or “GH16 SP” and is represented by SEQ ID NO:4, derived from the A. luchuensis glucanase polypeptide with SEQ ID NO: 23.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Introduced: The term “introduced” in the context of inserting a nucleic acid sequence into a cell, means “transfection”, “transformation” or “transduction,” as known in the art.

Isolated: The term “isolated” means a polypeptide, nucleic acid, cell, or other specified material or component that has been separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted polypeptide expressed in a host cell.

Mannanase: The term “mannanase” means a mannanase comprising a signal peptide at its N-terminal end, the mannanase having endo-1,4-beta-mannanase activity (EC 3.2.1.78). A non limiting example for a manannase is the Aspergillus luchuensis mannanase. The signal peptide sequence derived of the Aspergillus luchuensis mannanase is termed “GH26” or “GH26 SP” and is represented by SEQ ID NO: 2, derived from the A. luchuensis mannanase polypeptide with SEQ ID NO: 21.

Mature polypeptide: The term “mature polypeptide” means a polypeptide in its mature form following translation and any post-translational modifications such as N-terminal processing (e.g. removal of signal peptide), C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g. having a different C-terminal and/or N-terminal amino acid) as compared to another host cell expressing the same polynucleotide. Mature polypeptides of the invention may therefore have slight differences at the N- and/or C-terminal due to such differentiated expression by the host cell. A mature polypeptide having one or more amino acids absent from the N- and/or C-terminal may be considered to be a “fragment” of the full-length polypeptide.

In one aspect the mature polypeptide is amino acids 1 to 123 of SEQ ID NO:6. In some aspects, the mature polypeptide is amino acids 19 to 141 of SEQ ID NO:8 and amino acids 1 to 18 of SEQ ID NO:8 are a signal peptide.

In some aspects, the mature polypeptide is amino acids 22 to 144 of SEQ ID NO:10 and amino acids 1 to 21 of SEQ ID NO:10 are a signal peptide.

In one aspect the mature polypeptide is amino acids 1 to 411 of SEQ ID NO:12. In some aspects, the mature polypeptide is amino acids 19 to 429 of SEQ ID NO:14 and amino acids 1 to 18 of SEQ ID NO: 14 are a signal peptide.

In some aspects, the mature polypeptide is amino acids 22 to 432 of SEQ ID NO:16 and amino acids 1 to 21 of SEQ ID NO: 16 are a signal peptide.

Mature polypeptide coding sequence: The term “mature polypeptide coding sequence” means a polynucleotide that encodes a mature polypeptide having phytase activity, or an ALAB polypeptide which forms a functional regulatory subunit of the lactose synthase (LS) heterodimer.

In one aspect the mature polypeptide coding sequence is nucleotides 1 to 369 of SEQ ID NO:5. In one aspect, the mature polypeptide coding sequence is nucleotides 55 to 423 of SEQ ID NO: 7 and nucleotides 1 to 54 of SEQ ID NO: 7 encode a signal peptide. In one aspect, the mature polypeptide coding sequence is nucleotides 64 to 432 of SEQ ID NO: 9 and nucleotides 1 to 63 of SEQ ID NO: 9 encode a signal peptide.

In one aspect, the mature polypeptide coding sequence is nucleotides 110 to 478 of SEQ ID NO: 18, and nucleotides 1 to 63 of SEQ ID NO:18 encode a signal peptide, and nucleotides 64 to 109 of SEQ ID NO: 18 is an intron.

In one aspect, the mature polypeptide coding sequence is nucleotides 101 to 469 of SEQ ID NO: 19, and nucleotides 1 to 54 of SEQ ID NO: 19 encode a signal peptide, and nucleotides 55 to 100 of SEQ ID NO: 19 is an intron. In one aspect the mature polypeptide coding sequence is nucleotides 1 to 1233 of SEQ ID NO: 11. In one aspect, the mature polypeptide coding sequence is nucleotides 55 to 1287 of SEQ ID NO: 13 and nucleotides 1 to 54 of SEQ ID NO: 13 encode a signal. In one aspect, the mature polypeptide coding sequence is nucleotides 64 to 1296 of SEQ ID NO: 15 and nucleotides 1 to 63 of SEQ ID NO: 15 encode a signal peptide.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably.

Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

Obtained polypeptide/peptide/polynucleotide: The term “obtained” or “derived” when used in reference to a polynucleotide sequence, ALAB sequence, polypeptide sequence, mannanase sequence, glucanase sequence, phytase sequence, variant sequence or signal peptide sequence, means that the molecule originally has been isolated from the given source and that the molecule can either be utilized in its native sequence or that the molecule is modified by methods known to the skilled person.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Parent: The term “parent” means a polypeptide functioning as a signal peptide, or a polypeptide having phytase activity, or an ALAB polypeptide which forms a functional regulatory subunit of the lactose synthase (LS) heterodimer, to which an alteration is made to produce variants of the present invention. The parent may be a naturally occurring (wild-type) polypeptide or a variant or fragment thereof.

Phytase: In the present context, a preferred Phytase according to the invention is classified as belonging to the EC 3.1.3.26 group. The EC numbers refer to Enzyme Nomenclature 1992 from NC-IUBMB, Academic Press, San Diego, California, including supplements 1-5 published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650; respectively. The nomenclature is regularly supplemented and updated; see e.g. the World Wide Web at http://www.chem.qmw.ac.uk/iubmb/enzyme/index.html. A non-limiting example for a phytase is shown in SEQ ID NO:12.

Phytase activity: For the purpose of the present invention, phytase activity is determined by the liberation of inorganic phosphate from Na-phytate solution, wherein one phytase activity unit is the amount of enzyme which liberates 1 μmol inorganic phosphate per min from a 0.0051 M Na-phytate solution in 0.25 M Na-acetate, pH 5.5 and at 37° C. (Engelen, A. J., et al., 1994, “Simple and rapid determination of phytase activity”, J. AOAC Int. 77:760-764). Examples of activity unit names are: FYT, FTU and U. Phytase activity may be determined using the assay as described in Example 1 (“Determination of phytase activity”). In one aspect, the polypeptides of the present invention have at least 20%, e.g., at least 40%, at least 50%, at least 60%, at least 70%, at least 80%, at least 90%, at least 95%, or at least 100% of the phytase activity of SEQ ID NO:12. Detailed descriptions of how to detect phytase activity is given in the Examples under “pNP assay” and “FYT (B) assay”.

Specific activity is measured on highly purified samples (an SDS poly acryl amide gel should show the presence of only one component). The enzyme protein concentration may be determined by amino acid analysis, and the phytase activity in the units of FYT. Specific activity is a characteristic of the specific phytase variant in question, and it is calculated as the phytase activity measured in FYT units per mg phytase enzyme protein.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, polypeptide or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process.

Subsequence: The term “subsequence” means a polynucleotide having one or more nucleotides absent from the 5′ and/or 3′ end of a mature polypeptide coding sequence; wherein the subsequence encodes a fragment having phytase activity, or wherein the subsequence forms a functional regulatory subunit of the lactose synthase (LS) heterodimer.

Variant: The term “variant” means a polypeptide having phytase activity, or a polypeptide forming a functional regulatory subunit of the lactose synthase (LS) heterodimer, comprising a man-made mutation, i.e., a substitution, insertion (including extension), and/or deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### DETAILED DESCRIPTION OF THE INVENTION

The present invention is based on the surprising and inventive finding that expression of difficult-to-express proteins (alpha-lactalbumin and phytase) with signal peptides obtained from fungal polypeptides provides increased yield when expressed in fungal host cells.

Use of the signal peptides of the invention, an improved yield of both ALAB and phytase is achieved.

Notably, the increased expression was achieved using several, different fermentation protocols at different scales, including microtiter plates (MTP), shake flasks (SF), micro-bioreactors, and laboratory fermentation tanks.

### Polynucleotides

The present invention also relates to polynucleotides encoding a polypeptide of the present invention, as described herein.

The polynucleotide may be a genomic DNA, a cDNA, a synthetic DNA, a synthetic RNA, a mRNA, or a combination thereof. The polynucleotide may be cloned from a strain of Aspergillus, or a related organism and thus, for example, may be a polynucleotide sequence encoding a variant of the polypeptide of the invention.

In an embodiment, the polynucleotide is a subsequence encoding a fragment having phytase activity or a fragment which forms a functional regulatory subunit of the lactose synthase (LS) heterodimer of the present invention.

In one embodiment the polynucleotide encoding the signal peptide of the present invention is isolated from an Aspergillus cell, such as an Aspergillus luchuensis cell.

The polynucleotide may also be mutated by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2:95-107.

In an aspect, the polynucleotide is isolated.

In another aspect, the polynucleotide is purified.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

In a first aspect, the invention relates to a nucleic acid construct comprising:
a first polynucleotide encoding a signal peptide having a sequence identity of at least 80% to SEQ ID NO: 45, SEQ ID NO:2, SEQ ID NO:4, SEQ ID NO: 47, or SEQ ID NO: 49; and
a) a second polynucleotide encoding an alpha-lactalbumin (ALAB) polypeptide having a sequence identity of at least 70% to the polypeptide sequence of SEQ ID NO:6; or
b) a second polynucleotide encoding a polypeptide having phytase activity;
wherein the first polynucleotide and the second polynucleotide are operably linked in translational fusion.

In one embodiment, the second polynucleotide is located downstream from the first polynucleotide.

In one embodiment, the signal peptide is a naturally occurring signal peptide, or a functional fragment or functional variant of a naturally occurring signal peptide.

In one embodiment, the signal peptide is from a filamentous fungal glycosidase.

In one embodiment, the construct is further comprising a third polynucleotide downstream of the first polynucleotide and upstream of the second polynucleotide.

In one embodiment, the third polynucleotide is a non-coding intron.

In one embodiment, the third polynucleotide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO:17 (gtaagtaacatccactctgttctagtgccatgctgagattgtacag).

In one embodiment, the construct comprises a polynucleotide sequence with a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO: 18 or SEQ ID NO: 19.

In one embodiment, the nucleic acid construct further comprises a heterologous promoter, and wherein said promoter, the first polynucleotide, and the second polynucleotide, and optionally the third polynucleotide, are operably linked.

In another embodiment, the promoter is a P3 promoter or a P3-based promoter, preferably the heterologous promoter is a tandem promoter comprising the P3 promoter or is a tandem promoter derived from the P3 promoter.

In one embodiment, the promoter is operably linked to an mRNA stabilizer region; preferably the mRNA stabilizer region is the cryIIIA mRNA stabilizer region.

In one embodiment, the signal peptide is a naturally occurring signal peptide, or a functional fragment or functional variant of a naturally occurring signal peptide.

In one embodiment, the signal peptide is from a glycosidase (EC 3.2.1).

In one embodiment, the signal peptide is obtained from a mannanase polypeptide (EC 3.2.1.78).

In one embodiment, the signal peptide is obtained from a b-transglycosidase polypeptide (EC 2.4.1.-).

In one embodiment, the signal peptide is obtained from a chitin b-1,3/1,6-glucanosyltransferase polypeptide (EC 2.4.1.-) polypeptide.

In one embodiment, the signal peptide is obtained from an endo-b-1,3-glucanase polypeptide or laminarinase polypeptide (EC 3.2.1.39).

In one embodiment, the signal peptide is obtained from a polypeptide, such as a mannanase, transglycosidase, glycosyltransferase, laminarinase, or glucanase, expressed by a filamentous fungal host cell.

In one embodiment, the signal peptide is obtained from a polypeptide expressed by a Aspergillus host cell, such as an Aspergillus luchuensis.

In one embodiment, the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:1 or SEQ ID NO:3; most preferably the polynucleotide comprises, consists essentially of, or consists of the mature polypeptide coding sequence of SEQ ID NO:1, SEQ ID NO:3, SEQ ID NO: 44, SEQ ID NO:46, or SEQ ID NO:48.

In one embodiment, the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:1; most preferably the polynucleotide comprises, consists essentially of, or consists of the mature polypeptide coding sequence of SEQ ID NO:1.

In one embodiment, the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:3; most preferably the polynucleotide comprises, consists essentially of, or consists of the mature polypeptide coding sequence of SEQ ID NO:3.

In one embodiment, the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:44; most preferably the polynucleotide comprises, consists essentially of, or consists of the mature polypeptide coding sequence of SEQ ID NO:44.

In one embodiment, the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:46; most preferably the polynucleotide comprises, consists essentially of, or consists of the mature polypeptide coding sequence of SEQ ID NO:46.

In one embodiment, the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:48; most preferably the polynucleotide comprises, consists essentially of, or consists of the mature polypeptide coding sequence of SEQ ID NO:48.

In one embodiment, the signal peptide is obtained from a glycosidase expressed by an Aspergillus species selected from the group consisting of Aspergillus awamori, Aspergillus foetidus, Aspergillus fumigatus, Aspergillus japonicus, Aspergillus nidulans, Aspergillus niger, Aspergillus luchuensis, or Aspergillus oryzae.

In one embodiment, the e signal peptide is obtained from a glycosidase expressed by Aspergillus luchuensis.

In one embodiment, the signal peptide has a sequence identity of at least 85%, e.g. at least 90%, at least 91%, at least 92%, at least 93%, at least 94%, at least 95%, at least 96%, at least 97%, at least 98%, at least 99%, or 100%, to SEQ ID NO:2.

In one embodiment, the signal peptide comprises, consists essentially of, or consists of SEQ ID NO: 2.

In one embodiment, the signal peptide has a sequence identity of at least 85%, e.g. at least 90%, at least 91%, at least 92%, at least 93%, at least 94%, at least 95%, at least 96%, at least 97%, at least 98%, at least 99%, or 100%, to SEQ ID NO:4.

In one embodiment, the wherein the signal peptide comprises, consists essentially of, or consists of SEQ ID NO:4.

In one embodiment, the signal peptide has a sequence identity of at least 85%, e.g. at least 90%, at least 91%, at least 92%, at least 93%, at least 94%, at least 95%, at least 96%, at least 97%, at least 98%, at least 99%, or 100%, to SEQ ID NO:45.

In one embodiment, the wherein the signal peptide comprises, consists essentially of, or consists of SEQ ID NO:45.

In one embodiment, the signal peptide has a sequence identity of at least 85%, e.g. at least 90%, at least 91%, at least 92%, at least 93%, at least 94%, at least 95%, at least 96%, at least 97%, at least 98%, at least 99%, or 100%, to SEQ ID NO:47.

In one embodiment, the wherein the signal peptide comprises, consists essentially of, or consists of SEQ ID NO:47.

In one embodiment, the signal peptide has a sequence identity of at least 85%, e.g. at least 90%, at least 91%, at least 92%, at least 93%, at least 94%, at least 95%, at least 96%, at least 97%, at least 98%, at least 99%, or 100%, to SEQ ID NO:49.

In one embodiment, the wherein the signal peptide comprises, consists essentially of, or consists of SEQ ID NO:49.

In one embodiment, the signal peptide consists of the amino acid sequence of SEQ ID NO:45, SEQ ID NO: 2, SEQ ID NO:4, SEQ ID NO: 47, or SEQ ID NO: 49 with or without its C-terminal alanine, or a peptide fragment thereof that retains the ability to direct the polypeptide into or across a cell membrane.

In one embodiment, the N- and/or C-terminal end of the signal peptide has been extended by addition of one or more amino acids.

In one embodiment, the polynucleotide encoding the alpha-lactalbumin polypeptide has a sequence identity of at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:5; most preferably the polynucleotide comprises, consists essentially of, or consists of the mature polypeptide coding sequence of SEQ ID NO:5.

In one embodiment, the alpha-lactalbumin polypeptide is a bovine alpha-lactalbumin polypeptide.

In one embodiment, the alpha-lactalbumin polypeptide is a human alpha-lactalbumin polypeptide.

In one embodiment, the alpha-lactalbumin polypeptide has a sequence identity of at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to the mature polypeptide of SEQ ID NO:6.

In one embodiment, the alpha-lactalbumin polypeptide comprises, consists essentially of, or consists of the mature polypeptide of SEQ ID NO:6.

In one embodiment, the N- and/or C-terminal end of the alpha-lactalbumin polypeptide has been extended by addition of one or more amino acids.

In one embodiment, the polynucleotide encoding the polypeptide having phytase activity has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:11; most preferably the polynucleotide comprises, consists essentially of, or consists of the mature polypeptide coding sequence of SEQ ID NO:11.

In one embodiment, the polypeptide having phytase activity is a bacterial polypeptide or variant thereof.

In one embodiment, the polypeptide having phytase activity is EC 3.1.3.26.

In one embodiment, the polypeptide having phytase activity has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide of SEQ ID NO:12.

In one embodiment, the having phytase activity comprises, consists essentially of, or consists of the mature polypeptide of SEQ ID NO:12.

In one embodiment, the N- and/or C-terminal end of the polypeptide having phytase activity has been extended by addition of one or more amino acids.

It is expected that the invention will be just as effective when employing a signal peptide that is highly similar to the signal peptide disclosed in SEQ ID NO:45, SEQ ID NO:2, SEQ ID NO:4, SEQ ID NO: 47, and SEQ ID NO: 49, encoded by SEQ ID NO:1, SEQ ID NO:3, SEQ ID NO:44, SEQ ID NO: 46, and SEQ ID NO:48, respectively. One or more non-essential amino acids may, for example, be altered. Non-essential amino acids in a signal peptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant molecules are tested for signal peptide activity to identify amino acid residues that are critical to the activity of the molecule and residues that are non-essential. See also, Hilton et al., 1996, J. Biol. Chem. 271:4699-4708. The identity of essential and non-essential amino acids can also be inferred from an alignment with one or more related signal peptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g. Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

In one aspect, the signal peptide is a variant (i.e., functional variant) or fragment (i.e., functional fragment) of the signal peptide of SEQ ID NO:45, SEQ ID NO:2, SEQ ID NO:4, SEQ ID NO: 47, or SEQ ID NO: 49. In one aspect, the number of alterations in the signal peptide variant of the present invention is 1-10, e.g., 1-5, such as 1, 2, 3, 4, or 5 alterations. Alterations includes substitutions, insertions, and/or deletions at one or more (e.g., several) positions compared to the parent. A substitution means replacement of the amino acid occupying a position with a different amino acid; a deletion means removal of the amino acid occupying a position; and an insertion means adding an amino acid adjacent to and immediately following the amino acid occupying a position.

In a preferred embodiment, the signal peptide is a variant of the mature polypeptide of SEQ ID NO: 45, SEQ ID NO:2, SEQ ID NO:4, SEQ ID NO: 47, or SEQ ID NO: 49 comprising 1-10 alterations, e.g., 1-5, such as 1, 2, 3, 4, or 5 alterations, compared to SEQ ID NO:45, SEQ ID NO:2, SEQ ID NO:4, SEQ ID NO: 47, or SEQ ID NO: 49 respectively.

The first and second polynucleotide are operably linked in translational fusion. In the context of the present invention, the term “operably linked in translation fusion” means that the signal peptide encoded by the first polynucleotide and the polypeptide encoded by the second polynucleotide are encoded in frame and translated together as a single polypeptide. Preferably, following translation, the signal peptide is removed to provide the mature phytase polypeptide or the mature ALAB polypeptide. Alternatively, the signal peptide is not removed, or only removed partly to provide the mature ALAB polypeptide or the mature polypeptide having phytase activity and comprising at least a fragment of the signal peptide.

The first and second polynucleotide may be manipulated in a variety of ways to provide for expression of a variant. Manipulation of the polynucleotide prior to its insertion into a nucleic acid construct or expression vector may be desirable or necessary depending on the construct or vector. The techniques for modifying polynucleotides utilizing recombinant DNA methods are well known in the art.

Besides a signal peptide, the nucleic acid constructs of the invention may be operably linked to one or more further control sequences that direct the expression of the coding sequence in a suitable host cell under conditions compatible with the control sequences.

### Promoters

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

In one embodiment, the nucleic acid construct further comprises a heterologous promoter, and wherein said promoter, the first polynucleotide, and the second polynucleotide are operably linked. The promoter is orientated upstream of the first polynucleotide.

In an embodiment, the promoter is a heterologous promoter. Preferably, the promoter is a tandem promoter. More preferably, the promoter is a P3 promoter or a P3-based promoter.

Further suitable promoters for directing transcription of the polynucleotide of the present invention in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenbock, 2016, “Gene Expression Systems in Fungi: Advancements and Applications”, Fungal Biology.

For expression in a yeast host, examples of useful promoters are described by Smolke et al., 2018, “Synthetic Biology: Parts, Devices and Applications” (Chapter 6: Constitutive and Regulated Promoters in Yeast: How to Design and Make Use of Promoters in S. cerevisiae), and by Schmoll and Dattenbock, 2016, “Gene Expression Systems in Fungi: Advancements and Applications”, Fungal Biology.

In one embodiment, the promoter is a promoter, such as a P3 promoter, operably linked to an mRNA stabilizer region. Preferably, the mRNA stabilizer region is the cryIIIA mRNA stabilizer region.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471). Examples of mRNA stabilizer regions for fungal cells are described in Geisberg et al., 2014, Cell 156 (4): 812-824, and in Morozov et al., 2006, Eukaryotic Cell 5 (11): 1838-1846.

### Terminators

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenbock, 2016, “Gene Expression Systems in Fungi: Advancements and Applications”, Fungal Biology.

Preferred terminators for yeast host cells may be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, Yeast 8:423-488.

### Propeptides

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence. Additionally or alternatively, when both signal peptide and propeptide sequences are present, the polypeptide may comprise only a part of the signal peptide sequence and/or only a part of the propeptide sequence. Alternatively, the final or isolated polypeptide may comprise a mixture of mature polypeptides and polypeptides which comprise, either partly or in full length, a propeptide sequence and/or a signal peptide sequence.

### Regulatory Sequences

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In fungal systems, these regulatory sequences include the dihydrofolate reductase gene that is amplified in the presence of methotrexate, and the metallothionein genes that are amplified with heavy metals.

### Leader Sequences

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells may be obtained from the genes for Aspergillus oryzae TAKA amylase and Aspergillus nidulans triose phosphate isomerase.

Suitable leaders for yeast host cells may be obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e. by activating the transcription of the gene encoding the protein of interest by binding to its promoter, or indirectly, i.e. by activating the transcription of a further transcription factor which regulates the transcription of the gene encoding the protein of interest, such as by binding to the promoter of the further transcription factor. Suitable transcription factors for prokaryotic host cells are described in Seshasayee et al., Subcell Biochem 2011; 52:7-23, as well in Balleza et al., FEMS Microbiol Rev 2009, 33 (1): 133-151.

### Polyadenylation Sequences

The control sequence may also be a polyadenylation sequence, a sequence operably linked to the 3′-terminus of the polynucleotide which, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15:5983-5990.

### Expression Vectors

In a second aspect, the present invention also relates to recombinant expression vectors comprising a nucleic acid construct according to the first aspect. The expression vectors comprise a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Host Cells

In a third aspect, the invention relates to fungal host cells comprising in its genome:
a) a nucleic acid construct according to the first aspect; and/or
b) an expression vector according to the second aspect.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the polypeptide and its source. The polypeptide encoded by the introduced polynucleotide can be native or heterologous to the recombinant host cell. Also, at least one of the one or more control sequences can be heterologous to the polynucleotide encoding the polypeptide. The recombinant host cell may comprise a single copy, or at least two copies, e.g. three, four, five or more copies of the polynucleotide of the present invention.

In one embodiment, the host cell comprises two or more copies of the nucleic acid construct and/or the expression vector.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

Fungal cells may be transformed by a process involving protoplast-mediated transformation, Agrobacterium-mediated transformation, electroporation, biolistic method and shock-wave-mediated transformation as reviewed by Li et al., 2017, Microbial Cell Factories 16:168 and procedures described in EP 238023, Yelton et al., 1984, Proc. Natl. Acad. Sci. USA 81:1470-1474, Christensen et al., 1988, Bio/Technology 6:1419-1422, and Lubertozzi and Keasling, 2009, Biotechn. Advances 27:53-75. However, any method known in the art for introducing DNA into a fungal host cell can be used, and the DNA can be introduced as linearized or as circular polynucleotide.

The fungal host cell may be a yeast cell. “Yeast” as used herein includes ascosporogenous yeast (Endomycetales), basidiosporogenous yeast, and yeast belonging to the Fungi Imperfecti (Blastomycetes). For purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell. In a preferred embodiment, the yeast host cell is a Pichia or Komagataella cell, e.g., a Pichia pastoris cell (Komagataella phaffii).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, F Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

In one embodiment the host cell is an Aspergillus cell.

In another embodiment, the host cell is an Aspergillus niger cell.

In one embodiment, the host cell is an Aspergillus oryzae cell.

In an aspect, the host cell is isolated.

In one embodiment, the host cell comprises at least two copies of the nucleic acid construct and/or the expression vector, such as two copies, three copies, four copies or more than four copies.

In another aspect, the host cell is purified.

### Methods of Production

In a fourth aspect, the present invention also relates methods of producing an alpha-lactalbumin (ALAB) polypeptide, the method comprising:
a) cultivating a host cell according to the third aspect under conditions conducive for production of the ALAB polypeptide; and optionally
b) recovering the ALAB polypeptide.

In a fifth aspect, the present invention also relates to methods of producing a polypeptide having phytase activity, the method comprising:
a) cultivating a host cell according to the third aspect under conditions conducive for production of the polypeptide having phytase activity; and optionally
b) recovering the polypeptide having phytase activity.
   The host cell is cultivated in a nutrient medium suitable for production of the polypeptide using methods known in the art. For example, the cell may be cultivated by shake flask cultivation, or small-scale or large-scale fermentation (including continuous, batch, fed-batch, or solid-state, and/or microcarrier-based fermentations) in laboratory or industrial fermentors in a suitable medium and under conditions allowing the polypeptide to be expressed and/or isolated. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptide, including, but not limited to, the use of specific antibodies, formation of an enzyme product, disappearance of an enzyme substrate, or an assay determining the relative or specific activity of the polypeptide.

The polypeptide may be recovered from the medium using methods known in the art, including, but not limited to, collection, centrifugation, filtration, extraction, spray-drying, evaporation, or precipitation. In one aspect, a whole fermentation broth comprising the polypeptide is recovered. In another aspect, a cell-free fermentation broth comprising the polypeptide is recovered.

The polypeptide may be purified by a variety of procedures known in the art to obtain substantially pure polypeptides and/or polypeptide fragments (see, e.g., Wingfield, 2015, Current Protocols in Protein Science; 80 (1): 6.1.1-6.1.35; Labrou, 2014, Protein Downstream Processing, 1129:3-10).

In an alternative aspect, the polypeptide having phytase activity is not recovered. In one aspect the polypeptide having phytase activity is not recovered, but rather a host cell of the present invention expressing the polypeptide having phytase activity is used as a source of the variant.

### Fusion Polypeptide

In a sixth aspect, the invention relates to a fusion polypeptide, comprising
a) a signal peptide having at least 60% sequence identity to SEQ ID NO:45, SEQ ID NO:2, SEQ ID NO: 4, SEQ ID NO: 47, or SEQ ID NO: 49, and
b) an alpha-lactalbumin polypeptide having at least 60% sequence identity to SEQ ID NO: 6, or a polypeptide having phytase activity having at least 60% sequence identity to SEQ ID NO:8.

In one embodiment, the signal peptide is located upstream of the alpha-lactalbumin polypeptide or upstream of the polypeptide having phytase activity.

In one embodiment, the signal peptide is located at the N-terminal end of the alpha-lactalbumin polypeptide or at the N-terminal end of the polypeptide having phytase activity.

In a particular embodiment, the alpha-lactalbumin polypeptide is selected from the group consisting of:
(a) a polypeptide having at least 60% sequence identity to SEQ ID NO: 8;
(b) a polypeptide having at least 60% sequence identity to SEQ ID NO: 10;
(c) a polypeptide encoded by a polynucleotide having at least 60% sequence identity to the mature polypeptide coding sequence of SEQ ID NO: 7 or SEQ ID NO: 9;
(d) a polypeptide derived from SEQ ID NO: 8, SEQ ID NO: 10, SEQ ID NO: 18 or SEQ ID NO: 19, a mature polypeptide of SEQ ID NO: 8, SEQ ID NO:10, SEQ ID NO:18 or SEQ ID NO:19, having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(e) a polypeptide derived from the polypeptide of (a), (b), (c), or (d) wherein the N- and/or C-terminal end has been extended by addition of one or more amino acids;
(f) a fragment of the polypeptide of (a), (b), (c), (d), or (e), and
(g) the fragment of (f) wherein the polypeptide has 1-10 deletions at the N-terminal end.

In another embodiment, the polypeptide having phytase activity is selected from the group consisting of:
(a) a polypeptide having at least 60% sequence identity to SEQ ID NO: 14;
(b) a polypeptide having at least 60% sequence identity to SEQ ID NO: 16;
(c) a polypeptide encoded by a polynucleotide having at least 60% sequence identity to the mature polypeptide coding sequence of SEQ ID NO: 13 or SEQ ID NO: 15;
(d) a polypeptide derived from SEQ ID NO: 14 or SEQ ID NO: 16, a mature polypeptide of SEQ ID NO: 14 or SEQ ID NO:16, having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(e) a polypeptide derived from the polypeptide of (a), (b), (c), or (d) wherein the N- and/or C-terminal end has been extended by addition of one or more amino acids;
(f) a fragment of the polypeptide of (a), (b), (c), (d), or (e), and
(g) the fragment of (f) wherein the polypeptide has 1-10 deletions at the N-terminal end.

### Fermentation Broth Formulations or Cell Compositions

The present invention also relates to a fermentation broth formulation or a cell composition comprising a polypeptide having phytase activity. The fermentation broth product further comprises additional ingredients used in the fermentation process, such as, for example, cells (including, the host cells containing the nucleic acid constructs of the present invention which are used to produce the polypeptide having phytase activity), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiments, the fermentation broth formulation or the cell composition comprises a first organic acid component comprising at least one 1-5 carbon organic acid and/or a salt thereof and a second organic acid component comprising at least one 6 or more carbon organic acid and/or a salt thereof. In some embodiments, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In some embodiments, the killed cells and/or cell debris are removed from a cell-killed whole broth to provide a composition that is free of these components.

The fermentation broth formulation or cell composition may further comprise a preservative and/or anti-microbial (e.g., bacteriostatic) agent, including, but not limited to, sorbitol, sodium chloride, potassium sorbate, and others known in the art.

The cell-killed whole broth or cell composition may contain the unfractionated contents of the fermentation materials derived at the end of the fermentation. Typically, the cell-killed whole broth or cell composition contains the spent culture medium and cell debris present after the microbial cells (e.g., filamentous fungal cells) are grown to saturation, incubated under carbon-limiting conditions to allow protein synthesis. In some embodiments, the cell-killed whole broth or cell composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

The present invention is further described by the following examples that should not be construed as limiting the scope of the invention.

### Examples

### Example 1: Signal Peptide Library for Expression of Phytase

The phytase expression vectors comprising signal peptide variants JSP002, 010, 011 & 031 were prepared as follows.

The phytase expression plasmid is composed of phytase coding sequence (SEQ ID NO: 11) and the marker expression cassettes, and E. coli vector fragment. To prepare the expression plasmid for each signal peptide variant, two fragments were amplified. The first fragment (signal peptide fragment) with partial E. coli vector fragment and partial phytase expression cassette including promotor, signal peptide sequences and first 21 bases of 5′ end of mature phytase. Second fragment (mature fragment) contain partial phytase expression cassette including mature peptide sequence and terminator, marker expression cassette and the partial E. coli derived fragments. Two fragments have about 20 bp of overlapping sequences at their 5′ and 3′ ends and can be assembled by Gibson Assembly Kit. Obtained plasmid will harbor different signal peptides for phytase expression. For the amplification of mature fragment, overlap PCR was performed to amplify a fragment coding for each signal fragment and phytase mature fragment. It was inserted between the promoter and terminator of A. niger expression plasmid. The plasmid DNAs prepared introduced into A. niger hosts strains (host: C3085 and/or C5553). All strains comprise identical phytase gene copy number

SP GH13 with SEQ ID NO:25 is derived from A. niger endo-1,4-alpha amylase GH13.

SP cutinase with SEQ ID NO:27 is derived from Humicola insolens cutinase.

SP GH16 with SEQ ID NO:4 is derived from A. luchuensis endo-b-1,3-glucanase GH16 (SEQ ID NO: 23).

SP GH26 with SEQ ID NO:2 is derived from A. luchuensis endo-1,4-beta-mannanase GH26 (SEQ ID NO: 21).

TABLE 1
Signal sequences for phytase expression

| | |
|---|---|
| JSP004 | MKFFTTILSTASLVAALPAAVDSNHT |
| (SP cutinase) | PAAPELVAR (SEQ ID NO: 27) |
| JSP002 | MFAKLSLLSLLFSSAALG |
| (SP GH26) | (SEQ ID NO: 2) |
| JSP010 | MRLPLVSSTVALLSSASLVAA |
| (SP GH16) | (SEQ ID NO: 4) |
| JSP011 | MTPMVAWWSLFLYGLQVAAPALA |
| (SP GH13) | (SEQ ID NO: 25) |

TABLE 2
Primers

| | | Primer | |
|---|---|---|---|
| construct | Primer name | number | SEQ ID NO: |
| JSP002, 010, 011 | Mature frag_F | HA442 | 34 |
| | Mature frag_R | HA451 | 35 |
| | Signal frag_F | HA283 | 36 |
| JSP002 (SP GH26) | Signal frag_R | HA444 | 37 |
| | (JSP002) | | |
| JSP010 (SP GH16) | Signal frag_R | HA450 | 38 |
| | (JSP010) | | |
| JSP011 (SP GH13) | Signal frag_R | HA445 | 39 |
| | (JSP011) | | |
| JSP031 (SP GH72) | Mature frag_F | HA442 | 40 |
| | Mature frag_R | HA233 | 41 |
| | Signal frag_F | HA268 | 42 |
| JSP031 (SP GH72) | Signal frag R (JSP031) | HA489 | 43 |

### Example 2: SP GH16 and SP GH26 Show Increased Phytase Yields During MTP Cultivation

Transformants constructed as in example 1 were fermented in 96-well multi-titer-plate (MTP) containing ¼ YPG Ac, and 1% SBP at 30C for 3 days. The yield is detected by measuring phytase activity in culture supernatants using artificial substrate (described under section “pNP assay”). As can be seen from Table 3, increased phytase activities were measured using SP GH26 and SP GH16, compared to phytase activities using SP cutinase. In detail, SP GH26 showed 187% yield increase compared to SP cutinase, and SP GH16 showed 12% yield increase compared to SP cutinase.

TABLE 3
Relative phytase activities of signal variants in C3085 host

| Signal variants | Relative phytase activity (% to JSP004) |
|---|---|
| JSP004 (SP cutinase) | 100 |
| JSP002 (SP GH26) | 287 |
| JSP010 (SP GH16) | 112 |
| JSP011 (SP GH13) | 80 |

### Example 3: SP GH16 with Increased Phytase Yield During Lab Tank Fermentation

GH16 signal peptide was compared to SP GH13 by lab-tank fermented under the current standard conditions. The strains have genetically the same background, but integrated gene copy number of phytase signal variants are different as indicated in Table 4. Results are shown in Table 4, where JSP010 signal peptide GH16 showed significantly higher yield compared to SP GH13, i.e. 2.3-fold increase. The yield is measured by phytase activity using phytase as substrate (FYT (B) assay).

TABLE 4
Relative phytase yield during lab tank fermentation

| | | relative phytase activity |
|---|---|---|
| | phytase gene | (% to JSP011) normalized |
| | copy number | to gene copy number |
| JSP010 (SP GH16) | 3 | 233.7 |
| JSP011 (SP GH13) | 2 | 100 |

### Example 4: Signal Peptide Library for Expression of Bovine Alpha-Lactalbumin (ALAB)

Signal peptide variants (shown in table 5) for expression of bovine alpha-lactalbumin (ALAB, SEQ ID NO: 6) was constructed by 2 transformation steps. The first transformation (host C2552) was done to integrate an expression cassette of bovine alpha-lactalbumin, but an intron of an amylase containing PAM sequence is inserted in place of signal sequence. To the obtained strain, second transformation was performed. The target signal sequence fragments were amplified by PCR and integrated in between the promoter and mature sequence of bovine alpha-lactalbumin by CRISPR system. Obtained strains were bovine alpha-lactalbumin expressing strains under six different signal peptides.

SP GH72 with SEQ ID NO:29 is derived from A. niger beta-1,3-glucanosyltransferase GH72 with GPI-anchor.

SP GH16 with SEQ ID NO:4 is derived from A. luchuensis endo-b-1,3-glucanase GH16 (SEQ ID NO: 23).

SP GH26 with SEQ ID NO:2 is derived from A. luchuensis endo-1,4-beta-mannanase GH26 (SEQ ID NO: 21).

SP LYA1_4 with SEQ ID NO:45.

SP pepesin A with SEQ ID NO:47.

SP GH28_9 with SEQ ID NO:49.

TABLE 5
Signal sequences tested for ALAB expression

| | | |
|---|---|---|
| SP GH26 | JSP002 | MFAKLSLLSLLFSSAALG |
| | | (SEQ ID NO: 2) |
| SP GH16 | JSP010 | MRLPLVSSTVALLSSASLVAA |
| | | (SEQ ID NO: 4) |
| SP GH72 | JSP031 | MKFSATTLAGASLLLGRAIA |
| | | (SEQ ID NO: 29) |
| SP LYA1_4 | JSP017 | MKYAAALTAVAALAARAAA |
| | | (SEQ ID NO: 45) |
| SP pepesin A | JSP019 | MVVFSKTAALVLGLSSAVSA |
| | | (SEQ ID NO: 47) |
| SP GH28_9 | JSP008 | MHFLQNAFVAATMGAALAAA |
| | | (SEQ ID NO: 49) |

### Example 5: Increased Yield of Alpha-Lactalbumin Using SP GH26

Transformants JSP002 and JSP031 constructed as in example 4 were fermented by MTP or by buffled shake flasks (SF). The culture broth was centrifuged (3,500×g, 15 min) and the supernatant was used for yield evaluation by MALDI-TOF MS semi-quantification method.

### Semi-Quantification by MALDI-TOF MS

Yield of bovine alpha-lactalbumin was briefly measured by the intensity of MS spectrum relative to the reference signal. Results are summarized in Table 6. Accordingly, using SP GH26, ALAB expression increased by 91% compared to ALAB expression with SP GH72 (MTP). In shake flask cultivation, ALAB expression with SP GH26 was increased ca. 2.6-fold compared to ALAB expression with SP GH72.

TABLE 6
Relative ALAB yield using different signal sequences
and fermentation protocols (MTP & SF).

Relative ALAB yield (% to JSP031)

| Signal | MTP | SF |
|---|---|---|
| JSP002 (SP GH26) | 191% | 258% |
| JSP031 (SP GH72) | 100% | 100% |

### Example 6: SP GH16 and SP GH26 Result in Similar ALAB Yield in Lab Tank Fermentation

The JSP002 (SP GH26) signal peptide which showed superior expression compared to JSP031 SP in MTP and SF format of example 5, was compared to the JSP010 (SP GH16) signal peptide for the expression of ALAB. Therefore, ALAB expressing strains were re-constructed with A. niger host C6216 comprising three ALAB encoding gene copies, each with either SP GH26 or SP GH16 located upstream of the ALAB gene. The expression plasmids were prepared by overlap PCR of signal and mature alpha-lactalbumin (ALAB) fragments. Obtained strains were fermented in lab-tanks under the current standard conditions and yields were compared by band intensities on SDS-PAGE. As shown in Table 7, no significant ALAB yield difference was observed between two signal variants SP GH16 and SP GH26. Thus, both signal variants have significant yield increases compared to JSP031 from example 5, in which example SP GH26 showed 191% increased ALAB yield compared to JSP031 with SP from GH72.

TABLE 7
Relative ALAB yields using different signal sequence
in different fermentation protocols (SF & lab tank).

| | Lab Tank | SF (data from |
|---|---|---|
| | (Example 6) | Example 5) |
| | Relative yield | Relative yield |
| Signal | (% to JSP002) | (% to JSP002) |
| JSP002 (SP GH26) | 100 | 100 |
| JSP010 (SP GH16) | 98 | — |
| JSP031 (SP GH72) | — | 38.8 |

### Example 7: Increased ALAB Yield in MTP Cultivation

Additional signal peptide variants for expression of bovine ALAB (SEQ ID NO: 6) was constructed as described in Example 4. Signal peptide variants used in Example 7 are: JSP004 (control, cutinase SP with SEQ ID NO: 27), JSP008 (GH28_9 SP with SEQ ID NO: 49), JSP017 (LYA1_4 SP with SEQ ID NO: 45), JSP002 (SP GH 26, SEQ ID NO:2), JSP031 (SP GH72, SEQ ID NO: 29), and JSP019 (pepesin A=pepA SP with SEQ ID NO: 47). Constructed strains were cultivated in MTP (multi-titer plates). The culture broth was centrifuged (3,500×g, 15 min) and the supernatant was used for yield evaluation by SDS-PAGE. Culture supernatant was deglycosylated with EndoH and loaded on to SDS-PAGE gel. Intensity of bands corresponding to ALAB were compared to the intensity of ALAB fused to the cutinase SP (JSP004) used as control. The ALAB yield of the different SP variants relative to the ALAB yield using the cutinase SP is shown in table 8. All of the tested SP variants show increased ALAB yield relative to the SP cutinase. As shown in table 8, JSP017 and JSP019 resulted in an 342% and 472% ALAB yield, respectively.

TABLE 8

| Signal peptide variant | Relative ALAB yield [%] |
|---|---|
| JSP004 (SP cutinase, SEQ ID NO: 27) | 100.0 |
| JSP002 (SP GH26, SEQ ID NO: 2) | 172.1 |
| JSP008 (GH28_9, SEQ ID NO: 49) | 287.2 |
| JSP017 (SP LYA1-4, SEQ ID NO: 45) | 342.8 |
| JSP019 (SP pepA, SEQ ID NO: 47) | 472.7 |
| JSP031 (SP GH72, SEQ ID NO: 29) | 324.8 |

### Example 8: Increased ALAB Yield in Micro-Bioreactors

Aspergillus niger strains expressing ALAB fused to different signal peptides were cultivated in micro-bioreactors, each with a cultivation volume of 250 ml. The strains were constructed as described in Example 4, and grown in duplicates. Average ALAB yield was measured by size exclusion chromatography and is shown in Table 9, normalized to the JSP002 GH 26 SP with SEQ ID NO:2. As shown in table 9, ALAB yield is increased by 53% for the JSP017 signal peptide, relative to the JSP002 signal peptide.

TABLE 9

| Signal peptide variant | Relative ALAB yield [%] |
|---|---|
| JSP002 (SEQ ID NO: 2) | 100 |
| JSP017 (SEQ ID NO: 45) | 153 |
| JSP031 (SEQ ID NO: 29) | 141 |
| JSP019 (SEQ ID NO: 47) | 110 |

### Materials and Methods

Unless otherwise stated, DNA manipulation and transformation were performed using standard methods of molecular biology as described in Sambrook et al. (1989) Molecular cloning: A laboratory manual, Cold Spring Harbor lab., Cold Spring Harbor, NY; Ausubel, F. M. et al. (eds.) “Current protocols in Molecular Biology”, John Wiley and Sons, 1995; Harwood, C. R., and Cut-ting, S. M. (eds.) “Molecular Biological Methods for Bacillus”. John Wiley and Sons, 1990.

Purchased Material (E. coli and Kits)

E. coli DH5a (Toyobo) is used for plasmid construction and amplification. Amplified plasmids are recovered with Qiagen Plasmid Kit (Qiagen). Ligation is done with either Rapid DNA Dephos & Ligation Kit (Roche) or Gibson assembly kit (NEB) according to the manufactory instructions. Polymerase Chain Reaction (PCR) is carried out with KOD-Plus system (TOYOBO) or PrimeSTAR MAX DNA Polymerase (Takara). QIAquick™ Gel Extraction Kit (Qiagen) is used for the purification of PCR fragments and extraction of DNA fragment from agarose gel.

### Enzymes

Enzymes for DNA manipulations (e.g. restriction endonucleases, ligases etc.) are obtainable from New England Biolabs, Inc. and were used according to the manufacturer's instructions.

### Plasmids

The sequence for phytase harboring the Phytase from Citrobacterium braakii is described as SEQ ID NO: 11 (coding sequence) and SEQ ID NO:12 (Amino acid sequence).

### Microbial Strains

The expression host strain Aspergillus niger C2552 was isolated by Novozymes and is a derivative of Aspergillus niger NN049184 which was isolated from soil described in example 14 in WO2012/160093. C3085, C5553, C6242 are strains which can produce the glucoamylase (1,4-alpha-D-glucan glucohydrolase, EC 3.2.1.3) from Gloeophyllum sepiarium (Gs AMG).

### Medium

COVE trace metals solution was composed of 0.04 g of NaB4O7·10H2O, 0.4 g of CuSO4·5H2O, 1.2 g of FeSO4·7H2O, 0.7 g of MnSO4·H2O, 0.8 g of Na2MoO2·2H2O, 10 g of ZnSO4·7H2O, and deionized water to 1 liter.

50×COVE salts solution was composed of 26 g of KCl, 26 g of MgSO4·7H2O, 76 g of KH2PO4, 50 ml of COVE trace metals solution, and deionized water to 1 liter.

COVE medium was composed of 342.3 g of sucrose, 20 ml of 50×COVE salts solution, 10 ml of 1 M acetamide, 10 ml of 1.5 M CsCl2, 25 g of Noble agar, and deionized water to 1 liter.

COVEII plus 5-Fluorocytosine top agarose was composed of 34 g of sucrose, 20 ml of 50×COVE salts solution, 10 ml of 1 M acetamide, 2 ml of 5 g/L 5-Fluorocytosine, 10 g of low melt agarose, and deionized water to 1 liter.

COVE-N-GlyX plates were composed of 218 g of xylitol, 10 g of glycerol, 2.02 g of KNO3, 50 ml of COVE salts solution, 25 g of Noble agar, and deionized water to 1 liter.

STC buffer was composed of 0.8 M sorbitol, 25 mM Tris pH 8, and 25 mM CaCl2.

STPC buffer was composed of 40% PEG 4000 in STC buffer.

LB medium was composed of 10 g of tryptone, 5 g of yeast extract, 5 g of sodium chloride, and deionized water to 1 liter.

LB plus ampicillin plates were composed of 10 g of tryptone, 5 g of yeast extract, 5 g of sodium chloride, 15 g of Bacto agar, ampicilin at 100 μg per ml, and deionized water to 1 liter.

YPG medium was composed of 10 g of yeast extract, 20 g of Bacto peptone, 20 g of glucose, and deionized water to 1 liter.

SOC medium was composed of 20 g of tryptone, 5 g of yeast extract, 0.5 g of NaCl, 10 ml of 250 mM KCl, and deionized water to 1 liter.

TAE buffer was composed of 4.84 g of Tris Base, 1.14 ml of Glacial acetic acid, 2 ml of 0.5 M EDTA pH 8.0, and deionized water to 1 liter.

¼YPG Ac, 1% SBP was composed of 5.0 g/L glucose, 2.5 g/L yeast extract, 5 g/L peptone, 10 g/L soy bean powder, 5 ml/L 2M sodium acetate buffer, pH4.5 MSG is composed of 72 g Glycerol, 92 g Soybean powder (pH 6.0), water to 1 litre.

MU-1 glu is composed 260 g of glucose, 3 g of MgSO4·7H2O, 5 g of KH2PO4, 6 g of K2SO4, amyloglycosidase trace metal solution (pH 4.5), water to 1 liter.

Amyloglycosidase trace metal solution is 13.9 g/L of FeSO4·7H2O, 13.56 g/L of MnSO4.5H2O, 6.8 g/L of ZnCl2, 2.5 g/L of CuSO4.5H2O, 0.24 g/L of NiCl2·6H2O, 3 g/L of Citric acid H2O.

Transformation of Aspergillus niger

Transformation of Aspergillus species can be achieved using the general methods for yeast transformation.

Aspergillus niger host strain was inoculated to 100 ml of YPG medium supplemented with 10 mM uridine and incubated for 16 h at 32° C. at 80 rpm. Pellets were collected and washed with 0.6 M KCl, and resuspended 20 ml 0.6 M KCl containing a commercial beta-glucanase product (GLUCANEX™, Novozymes A/S, Bagsværd, Denmark) at a final concentration of 20 mg per ml. The suspension was incubated at 32° C. at 80 rpm until protoplasts were formed, and then washed twice with STC buffer. The protoplasts were counted with a hematometer and resuspended and adjusted in an 8:2:0.1 solution of STC:STPC:DMSO to a final concentration of 2.5×107 protoplasts/ml. Approximately 4 μg of plasmid DNA was added to 100 μl of the protoplast suspension, mixed gently, and incubated on ice for 30 minutes. One ml of SPTC was added and the protoplast suspension was incubated for 20 minutes at 37° C. After the addition of 10 ml of 50° C. Cove top agarose, the reaction was poured onto Cove agar plates and the plates were incubated at 30° C. for 3 days. COVEII plus 5-Fluorocytosine top agarose was overlayed to the transformation plates for counter-selection of the strains.

### PCR Amplifications

Polymerase Chain Reaction (PCR) was carried out with KOD plus neo [ToYoBo] or PrimeSTAR Max DNA polymerase [TaKaRa]. KOD plus reaction mix is shown in table 10. KOD plus PCR cycle is shown in table 11. PrimeSTAR reaction mix is shown in table 12, and corresponding PCR cycle is shown in table 13.

TABLE 10
KOD plus neo-PCR reaction mix:

| | |
|---|---|
| Template DNA | 1.0 mL |
| 5x buffer | 5.0 mL |
| dNTP | 5.0 mL |
| MgSO4 | 3.0 mL |
| KOD plus neo | 1.0 mL |
| Forward primer (10 μM) | 1.5 mL |
| Reverse primer (10 μM) | 1.5 mL |
| water | Up to 50 mL |

TABLE 11
KOD plus neo-PCR program

| | | | |
|---|---|---|---|
| Step1 | 94° C. | 2 | min |
| Step2 | 94° C. | 10 | sec |
| Step3 | 55° C. | 30 | sec |

| | | |
|---|---|---|
| Step4 | 68° C. | 1 kbp/1 min |

Repeat step 2-4, ×30

| | | |
|---|---|---|
| Step5 | 10° C. | hold |

TABLE 12
PrimeSTAR Max DNA polymerase PCR reaction mix:

| | |
|---|---|
| Template DNA | 1.0 mL |
| 2 × PrimeSTAR Max DNA polymerase mix | 55.0 mL |
| Forward primer (10 μM) | 1.5 mL |
| Reverse primer (10 μM) | 1.5 mL |
| water | Up to 50 mL |

TABLE 13
PrimeSTAR Max DNA polymerase PCR program

| | | | | |
|---|---|---|---|---|
| Step1 | 96° | C. | 2 | min |
| Step2 | 96° | C. | 15 | sec |
| Step3 | Tm-5° | C. | 30 | sec |

| | | | |
|---|---|---|---|
| Step4 | 72° | C. | 1 kbp/10 sec |

Repeat step 2-4, ×35

| | | | |
|---|---|---|---|
| Step5 | 10° | C. | hold |

### MTP Fermentation

Spores of the selected transformants were inoculated in 0.45 mL of ¼YPG Ac, 1% SBP in 96 well MTP and cultivated at 30° C. for 3 days at 900 rpm.

### Shake Flask (SF) Fermentation

Spores are inoculated to 100 ml of MSG media in buffled flask and fermented on a rotary shaking table at 220 rpm, 30° C. Ten-milli liters of cultured broth was transferred to 100 ml MU1-glu with 4 ml 50% urea in 500 ml baffled flasks and further fermented for 3 to 5 days.

### Lab Tank Fermentation

Fermentation was done as fed-batch fermentation (H. Pedersen 2000, Appl Microbiol Biotechnol, 53:272-277). Selected strains were pre-cultured in liquid media then grown mycelia were transferred to the tanks for further cultivation of protein production. Cultivation was done at pH 4 to 7, 30 to 34° C., for 6~8 days with the feeding of glucose and ammonium without over-dosing. Culture supernatant after centrifugation was used for yield evaluation.

### pNP Assay

Dilute cultured sup and purified standard samples to appropriate concentrations by 100 mM Sodium acetate buffer pH5.5. Start enzymatic reaction by mixing 10 μL of diluted samples and 100 uL of substrate (10 mM p-nitrophenyl-phosphate, disodium in 100 mM Sodium acetate pH5.5) in 96-well plate. After incubation at room temperature for 18 min, add 80 μl of stop solution (0.5M NA2CO₃, pH>11), hold for 2 min and measure absorbance at 405 nm. Enzyme activity was calculated from standard curve and indicated as relative activity to the reference.

### FYT(B) Assay

Phytase activity was measured as FYT (B) (Phytase (Braakii) Units), relative to an enzyme standard of a declared strength.

The samples and standard phytase reacts with sodium phytase (phytic acid dodeca sodium salt C₆H₆O₂₄P₆Na₁₂) and releases inorganic phosphate. Catalytic reaction parameters and conditions are shown in table 14. This phosphate is determined spectrophometically from a yellow complex formed by an acidic complex reagent containing molybdate/vanadate. The yellow complex is measured spectrophotometrically at a wavelength of 405 nm. The rate of phosphate release can be observed by Konelab (Thermo Fisher Scientific). Colorimetric reaction parameters and conditions are shown in Table 15. Table 16 shows reaction buffers and reagent compositions.

TABLE 14
Catalytic reaction

| Parameter | Reaction conditions |
|---|---|
| Temperature [° C.] | 37 |
| pH | 5.5 at room temperature |
| Substrate conc. | 5.0 mM |
| Enzyme conc. | 5.553 mFYT(B)/mL |
| Reaction time [sec.] | 1800 |
| Wave length [nm] | 405 nm (Side measurement at 380 nm subtracted) |

TABLE 15
Colorimetric reaction

| Parameter | Reaction conditions |
|---|---|
| Temperature [° C.] | 37 |
| Color reagent conc | Ammonium heptamolybdate tetrahydrate 8 mM |
| | Ammonium metavanadate 0.024% |
| | Nitric acid 4.4% |
| | Ammonium hydroxide solution 0.024% |
| Reaction time before | 300 |
| measuremant [sec.] | |
| Wave length [nm] | 405 nm (Side measurement at 380 nm subtracted) |

TABLE 16
Reaction buffer and reagents composition

| | |
|---|---|
| Dilution buffer | 0.25M CH₃COONa, 3 H₂O, pH 5.5 |
| | 0.01% (w/v) TWEEN ® 20 |
| Substrate | 0.25M CH₃COONa 3H₂O, pH 5.5 |
| | 7.5 mM Phytic acid dodecasodium salt hydrate |
| Stop reagent | 0.02M Ammonium heptamolybdate tetrahydrate |
| | 10% Nitric acid |
| | 0.06% Ammonium hydroxide solution |
| | 0.06% Ammonium metavanadate |

The activity of the enzyme samples is determined relative to the standard curve.

Calculation was conducted as follows:
Activity FYT (B)/g=(S×V×F)/W
S=Reading from the standard curve in FYT (B)/ml
V=Volume of the measuring flask used in mL
F=Dilution factor for second dilution
W=Weight of sample in g

### Semi-Quantification by MALDI-TOF MS

Diluted culture supernatant was mixed with an internal standard protein (e.g EndoH) and MS spectrum was taken. The ratio of signal intensities of target (bovine alpha-lactalbumin) and reference (internal standard) was recorded as rough expression level of target. Standard curve was prepared using series of diluted purified bovine alpha-lactalbumin.

### Size Exclusion Chromatography (SEC)

Quantification of ALAB was carried out by size exclusion chromatography. SEC protocol is modified from Pinho et al. Journal of Dairy Research, 79 (2) 2012.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### LIST OF EMBODIMENTS

The invention is further defined by the following numbered embodiments:
[1] A nucleic acid construct comprising:
   a first polynucleotide encoding a signal peptide having a sequence identity of at least 80% to SEQ ID NO: 45, SEQ ID NO:2, SEQ ID NO:4, SEQ ID NO: 47, or SEQ ID NO: 49; and
   a) a second polynucleotide encoding an alpha-lactalbumin (ALAB) polypeptide having a sequence identity of at least 70% to the polypeptide sequence of SEQ ID NO:6; or
   b) a second polynucleotide encoding a polypeptide having phytase activity;
   wherein the first polynucleotide and the second polynucleotide are operably linked in translational fusion.
[2] The nucleic acid construct according to embodiment 1, wherein the second polynucleotide is located downstream from the first polynucleotide.
[3] The nucleic acid construct according to any of embodiment 1 or 2, wherein the signal peptide is a naturally occurring signal peptide, or a functional fragment or functional variant of a naturally occurring signal peptide.
[4] The nucleic acid construct according to any of embodiments 1 to 3, wherein the signal peptide is from a filamentous fungal glycosidase.
[4a] The nucleic acid construct according to any of embodiments 1 to 4, further comprising a third polynucleotide downstream of the first polynucleotide and upstream of the second polynucleotide.
[4b] The nucleic acid construct according to any previous embodiment, wherein the third polynucleotide is a non-coding intron.
[4c] The nucleic acid construct according to any previous embodiment, wherein the third polynucleotide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO: 17 (gtaagtaacatccactctgttctagtgccatgctgagattgtacag).
[4d] The nucleic acid construct according to any previous embodiment, comprising a polynucleotide sequence with a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO: 18 or SEQ ID NO:19.
[5] The nucleic acid construct according to any previous embodiment, wherein the nucleic acid construct further comprises a heterologous promoter, and wherein said promoter, the first polynucleotide, the second polynucleotide, and optionally the third polynucleotide, are operably linked.
[6] The nucleic acid construct according to any previous embodiment, wherein the promoter is a P3 promoter or a P3-based promoter, preferably the heterologous promoter is a tandem promoter comprising the P3 promoter or is a tandem promoter derived from the P3 promoter.
[7] The nucleic acid construct according to any previous embodiment, wherein the promoter is operably linked to an mRNA stabilizer region; preferably the mRNA stabilizer region is the cryIIIA mRNA stabilizer region.
[8] The nucleic acid construct according to any of the preceding embodiments, wherein the signal peptide is a naturally occurring signal peptide, or a functional fragment or functional variant of a naturally occurring signal peptide.
[9] The nucleic acid construct according to any of the preceding embodiments, wherein the signal peptide is from a glycosidase (EC 3.2.1).
[10] The nucleic acid construct according to any of the preceding embodiments, wherein the signal peptide is obtained from a mannanase polypeptide (EC 3.2.1.78).
[11] The nucleic acid construct according to any of the preceding embodiments, wherein the signal peptide is obtained from a b-transglycosidase polypeptide (EC 2.4.1.-).
[12] The nucleic acid construct according to any of the preceding embodiments, wherein the signal peptide is obtained from a chitin b-1,3/1,6-glucanosyltransferase polypeptide (EC 2.4.1.-) polypeptide.
[13] The nucleic acid construct according to any of the preceding embodiments, wherein the signal peptide is obtained from an endo-b-1,3-glucanase polypeptide or laminarinase polypeptide (EC 3.2.1.39).
[14] The nucleic acid construct according to any of the preceding embodiments, wherein the signal peptide is obtained from a polypeptide, such as a mannanase, transglycosidase, glycosyltransferase, laminarinase, or glucanase, expressed by a filamentous fungal host cell.
[15] The nucleic acid construct according to any of the preceding embodiments, wherein the signal peptide is obtained from a polypeptide expressed by a Aspergillus host cell, such as an Aspergillus luchuensis.
[16] The nucleic acid construct according to any of the preceding embodiments, wherein the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:1, SEQ ID NO: 3, SEQ ID NO: 44, SEQ ID NO:46, or SEQ ID NO:48; most preferably the polynucleotide comprises, consists essentially of, or consists of the mature polypeptide coding sequence of SEQ ID NO:1, SEQ ID NO: 3, SEQ ID NO:44, SEQ ID NO:46, or SEQ ID NO:48.
[17] The nucleic acid construct according to any of the preceding embodiments, wherein the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:1; most preferably the polynucleotide comprises, consists essentially of, or consists of the mature polypeptide coding sequence of SEQ ID NO:1.
[18a] The nucleic acid construct according to any of the preceding embodiments, wherein the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:3; most preferably the polynucleotide comprises, consists essentially of, or consists of the mature polypeptide coding sequence of SEQ ID NO:3.
[18b] The nucleic acid construct according to any of the preceding embodiments, wherein the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:44; most preferably the polynucleotide comprises, consists essentially of, or consists of the mature polypeptide coding sequence of SEQ ID NO:44.
[18c] The nucleic acid construct according to any of the preceding embodiments, wherein the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:46; most preferably the polynucleotide comprises, consists essentially of, or consists of the mature polypeptide coding sequence of SEQ ID NO:46.
[18d] The nucleic acid construct according to any of the preceding embodiments, wherein the first polynucleotide encoding the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:48; most preferably the polynucleotide comprises, consists essentially of, or consists of the mature polypeptide coding sequence of SEQ ID NO:48.
[19] The nucleic acid construct according to any of the preceding embodiments, wherein the signal peptide is obtained from a glycosidase expressed by an Aspergillus species selected from the group consisting of Aspergillus awamori, Aspergillus foetidus, Aspergillus fumigatus, Aspergillus japonicus, Aspergillus nidulans, Aspergillus niger, Aspergillus luchuensis, or Aspergillus oryzae.
[20] The nucleic acid construct according to any of the preceding embodiments, wherein the signal peptide is obtained from a glycosidase expressed by Aspergillus luchuensis.
[21] The nucleic acid construct according to any of the preceding embodiments, wherein the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO: 2.
[22a] The nucleic acid construct according to any of the preceding embodiments, wherein the signal peptide comprises, consists essentially of, or consists of SEQ ID NO:2.
[22b] The nucleic acid construct according to any of the preceding embodiments, wherein the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO: 45.
[22c] The nucleic acid construct according to any of the preceding embodiments, wherein the signal peptide comprises, consists essentially of, or consists of SEQ ID NO:45.
[22d] The nucleic acid construct according to any of the preceding embodiments, wherein the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO: 47.
[22e] The nucleic acid construct according to any of the preceding embodiments, wherein the signal peptide comprises, consists essentially of, or consists of SEQ ID NO:47.
[22f] The nucleic acid construct according to any of the preceding embodiments, wherein the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO: 49.
[22 g] The nucleic acid construct according to any of the preceding embodiments, wherein the signal peptide comprises, consists essentially of, or consists of SEQ ID NO:49.
[23] The nucleic acid construct according to any of the preceding embodiments, wherein the signal peptide has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to SEQ ID NO: 4.
[24] The nucleic acid construct according to any of the preceding embodiments, wherein the signal peptide comprises, consists essentially of, or consists of SEQ ID NO:4.
[24a] The nucleic acid construct according to any preceding embodiments, wherein the signal peptide consists of the amino acid sequence of SEQ ID NO:45, SEQ ID NO:2, SEQ ID NO:4, SEQ ID NO: 47, or SEQ ID NO: 49 with or without its C-terminal alanine, or a peptide fragment thereof that retains the ability to direct the polypeptide into or across a cell membrane.
[25] The nucleic acid construct according to any of the preceding embodiments, wherein the N- and/or C-terminal end of the signal peptide has been extended by addition of one or more amino acids.
[26] The nucleic acid construct according to any of embodiments 1 to 25, wherein the signal peptide is a fragment of the signal peptides of any of embodiments 1 to 25.
[27] The nucleic acid construct according to any of the preceding embodiments, wherein polynucleotide encoding the alpha-lactalbumin polypeptide has a sequence identity of at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO:5; most preferably the polynucleotide comprises, consists essentially of, or consists of the mature polypeptide coding sequence of SEQ ID NO:5.
[28] The nucleic acid construct according to any of the preceding embodiments, wherein the alpha-lactalbumin polypeptide is a bovine alpha-lactalbumin.
[29] The nucleic acid construct according to any of the preceding embodiments, wherein the alpha-lactalbumin is a human alpha-lactalbumin.
[30] The nucleic acid construct according to any preceding embodiments, wherein the alpha-lactalbumin polypeptide has a sequence identity of at least 60%, e.g. at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, to the mature polypeptide of SEQ ID NO:6.
[31] The nucleic acid construct according to embodiment 30, wherein the alpha-lactalbumin polypeptide comprises, consists essentially of, or consists of the mature polypeptide of SEQ ID NO:6.
[32] The nucleic acid construct according to any of the preceding embodiments, wherein the N- and/or C-terminal end of the alpha-lactalbumin polypeptide has been extended by addition of one or more amino acids.
[33] The nucleic acid construct according to any of the preceding embodiments, wherein polynucleotide encoding the polypeptide having phytase activity has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO: 11; most preferably the polynucleotide comprises, consists essentially of, or consists of the mature polypeptide coding sequence of SEQ ID NO:11.
[34] The nucleic acid construct according to any of the preceding embodiments, wherein the polypeptide having phytase activity is a bacterial polypeptide or variant thereof.
[35] The nucleic acid construct according to any of the preceding embodiments, wherein the polypeptide having phytase activity is EC 3.1.3.26.
[36] The nucleic acid construct according to any preceding embodiments, wherein the polypeptide having phytase activity has a sequence identity of at least 80%, e.g. at least 85%, at least 90%, at least 91%, at least 92%, at least 93%, at least 94%, at least 95%, at least 96%, at least 97%, at least 98%, at least 99%, or 100%, to the mature polypeptide of SEQ ID NO: 12.
[37] The nucleic acid construct according to embodiment 36, wherein the polypeptide having phytase activity comprises, consists essentially of, or consists of the mature polypeptide of SEQ ID NO:12.
[38] The nucleic acid construct according to any of the preceding embodiments, wherein the N- and/or C-terminal end of the polypeptide having phytase activity has been extended by addition of one or more amino acids.
[39] The nucleic acid construct according to any of embodiments 1 to 38, wherein the signal peptide is a fragment of the signal peptides of any of embodiments 1 to 38.
[40] An expression vector comprising a nucleic acid construct according to any of embodiments 1 to 39.
[41] A fungal host cell comprising in its genome:
   a) a nucleic acid construct according to any of embodiments 1 to 39; and/or
   b) an expression vector according to embodiment 40.
[42] The host cell of embodiment 41, wherein the fungal host cell is a filmentous fungal host cell, e.g., an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell, in particular, an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.
[43] The host cell of any of embodiments 41 to 42, wherein the host cell is an Aspergillus cell.
[44] The host cell of any of embodiments 41 to 43, wherein the host cell is an Aspergillus niger or Aspergillus oryzae host cell.
[45] The host cell of any of embodiments 41 to 44, wherein the host cell comprises at least two copies of the nucleic acid construct and/or the expression vector, such as two copies, three copies, four copies or more than four copies.
[46] A method of producing a alpha-lactalbuming (ALAB) polypeptide, the method comprising:
   a) cultivating a host cell according to any of embodiments 41 to 45 under conditions conducive for production of the ALAB polypeptide; and optionally
   b) recovering the ALAB polypeptide.
[47] A method of producing a polypeptide having phytase activity, the method comprising:
   a) cultivating a host cell according to any of embodiments 41 to 45 under conditions conducive for production of the polypeptide having phytase activity; and optionally
   b) recovering the polypeptide having phytase activity.
[48] A fusion polypeptide, comprising
   a) a signal peptide having at least 60% sequence identity to SEQ ID NO:45, SEQ ID NO:2, SEQ ID NO: 4, SEQ ID NO: 47, or SEQ ID NO: 49, and
   b) an alpha-lactalbumin polypeptide having at least 60% sequence identity to SEQ ID NO: 6, or a polypeptide having phytase activity having at least 60% sequence identity to SEQ ID NO:8.
[49] The fusion polypeptide according to embodiment 48, wherein the signal peptide is located upstream of the alpha-lactalbumin polypeptide or upstream of the polypeptide having phytase activity.
[50] The fusion polypeptide according to any of embodiments 48 to 49, wherein the signal peptide is located at the N-terminal end of the alpha-lactalbumin polypeptide or at the N-terminal end of the polypeptide having phytase activity.
[51] The fusion polypeptide according to any of embodiments 48 to 50, wherein the alpha-lactalbumin polypeptide is selected from the group consisting of:
   (a) a polypeptide having at least 60% sequence identity to SEQ ID NO: 8;
   (b) a polypeptide having at least 60% sequence identity to SEQ ID NO: 10;
   (c) a polypeptide encoded by a polynucleotide having at least 60% sequence identity to the mature polypeptide coding sequence of SEQ ID NO: 7 or SEQ ID NO: 9;
   (d) a polypeptide derived from SEQ ID NO: 8, SEQ ID NO: 10, SEQ ID NO: 18 or SEQ ID NO:19, a mature polypeptide of SEQ ID NO: 8, SEQ ID NO:10, SEQ ID NO:18 or SEQ ID NO:19, having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (e) a polypeptide derived from the polypeptide of (a), (b), (c), or (d) wherein the N- and/or C-terminal end has been extended by addition of one or more amino acids;
   (f) a fragment of the polypeptide of (a), (b), (c), (d), or (e), and
   (g) the fragment of (f) wherein the polypeptide has 1-10 deletions at the N-terminal end.
[52] The fusion polypeptide according to any of embodiments 48 to 50, wherein the polypeptide having phytase activity is selected from the group consisting of:
   (a) a polypeptide having at least 60% sequence identity to SEQ ID NO: 14;
   (b) a polypeptide having at least 60% sequence identity to SEQ ID NO: 16;
   (c) a polypeptide encoded by a polynucleotide having at least 60% sequence identity to the mature polypeptide coding sequence of SEQ ID NO: 13 or SEQ ID NO: 15;
   (d) a polypeptide derived from SEQ ID NO: 14 or SEQ ID NO:16, a mature polypeptide of SEQ ID NO: 14 or SEQ ID NO: 16, having 1-30 alterations, e.g., substitutions, deletions and/or insertions at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
   (e) a polypeptide derived from the polypeptide of (a), (b), (c), or (d) wherein the N- and/or C-terminal end has been extended by addition of one or more amino acids;
   (f) a fragment of the polypeptide of (a), (b), (c), (d), or (e), and
   (g) the fragment of (f) wherein the polypeptide has 1-10 deletions at the N-terminal end.

1. A nucleic acid construct comprising:
a first polynucleotide encoding a signal peptide having a sequence identity of at least 80% to SEQ ID NO: 45, SEQ ID NO: 2, SEQ ID NO: 4, SEQ ID NO: 47, or SEQ ID NO: 49; and
a second polynucleotide encoding an alpha-lactalbumin (ALAB) polypeptide having a sequence identity of at least 70% to the polypeptide sequence of SEQ ID NO: 6;
wherein the first polynucleotide and the second polynucleotide are operably linked in translational fusion.

2. The nucleic acid construct according to claim 1, wherein the signal peptide has a sequence identity of at least 90% to SEQ ID NO: 45.

3. The nucleic acid construct according to claim 1, wherein the signal peptide has a sequence identity of at least 90% to SEQ ID NO: 2.

4. The nucleic acid construct according to claim 1, wherein the signal peptide consists of the amino acid sequence of SEQ ID NO: 2 or SEQ ID NO: 45 with or without its C-terminal alanine, or a peptide fragment thereof that retains the ability to direct the polypeptide into or across a cell membrane.

5. The nucleic acid construct according to claim 1, wherein the alpha-lactalbumin polypeptide has a sequence identity of at least 85% to the mature polypeptide of SEQ ID NO: 6.

6. An expression vector comprising a nucleic acid construct according to claim 1.

7. A fungal host cell comprising in its genome
an expression vector according to claim 6.

8. A method of producing an alpha-lactalbumin (ALAB) polypeptide, the method comprising:
a) cultivating a host cell according to claim 7 under conditions conducive for production of the ALAB polypeptide; and optionally
b) recovering the ALAB polypeptide.

9. A fusion polypeptide, comprising:
a signal peptide having at least 85% sequence identity to SEQ ID NO: 45, SEQ ID NO: 2, SEQ ID NO: 4, SEQ ID NO: 47, or SEQ ID NO: 49, and
an alpha-lactalbumin polypeptide having at least at least 85% sequence identity to SEQ ID NO: 6.

10. The nucleic acid construct according to claim 1, wherein the signal peptide has a sequence according to SEQ ID NO: 45.

11. The nucleic acid construct according to claim 1, wherein the signal peptide has a sequence according to SEQ ID NO: 2.

12. The nucleic acid construct according to claim 1, wherein the alpha-lactalbumin polypeptide has a sequence according to the mature polypeptide of SEQ ID NO: 6.

13. The fusion polypeptide according to claim 9,
wherein the signal peptide has a sequence according to SEQ ID NO: 45, SEQ ID NO: 2, SEQ ID NO: 4, SEQ ID NO: 47, or SEQ ID NO: 49, and
wherein the alpha-lactalbumin polypeptide has a sequence according to SEQ ID NO: 6.


SEQUENCE LISTING (49 sequences)

SEQ ID NO: 1 - DNA, 54 bp, Aspergillus luchuensis
  source 1..54: mol_type = genomic DNA
atgttcgccaaattgtccctcctttcgcttcttttcagctctgctgcgctgggc

SEQ ID NO: 2 - protein, 18 aa, Aspergillus luchuensis
MFAKLSLLSLLFSSAALG

SEQ ID NO: 3 - DNA, 63 bp, Aspergillus luchuensis
  source 1..63: mol_type = genomic DNA
atgagacttccgcttgttagctcgacagtcgctcttctttccagcgccagcttagtggcagcg

SEQ ID NO: 4 - protein, 21 aa, Aspergillus luchuensis
MRLPLVSSTVALLSSASLVAA

SEQ ID NO: 5 - DNA, 369 bp, synthetic construct
  source 1..369: mol_type = other DNA
gagcaactcaccaagtgcgaggtcttccgcgagctcaaggacctcaagggctacggcggcgtctccctccccgagtgggtctgcaccaccttccacacctccggctacgacacccaagccatcgtccaaaacaacgactccaccgagtacggcctcttccaaatcaacaacaagatctggtgcaaggacgaccaaaacccccactcctccaacatctgcaacatctcctgcgacaagttcctcgacgacgacctcaccgacgacatcatgtgcgtcaagaagatcctcgacaaggtcggcatcaactactggctcgcccacaaggccctctgctccgagaagctcgaccaatggctctgcgagaagctc

SEQ ID NO: 6 - protein, 123 aa, synthetic construct
EQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 7 - DNA, 423 bp, synthetic construct
  source 1..423: mol_type = other DNA
  sig_peptide 1..54
atgttcgccaaattgtccctcctttcgcttcttttcagctctgctgcgctgggcgagcaactcaccaagtgcgaggtcttccgcgagctcaaggacctcaagggctacggcggcgtctccctccccgagtgggtctgcaccaccttccacacctccggctacgacacccaagccatcgtccaaaacaacgactccaccgagtacggcctcttccaaatcaacaacaagatctggtgcaaggacgaccaaaacccccactcctccaacatctgcaacatctcctgcgacaagttcctcgacgacgacctcaccgacgacatcatgtgcgtcaagaagatcctcgacaaggtcggcatcaactactggctcgcccacaaggccctctgctccgagaagctcgaccaatggctctgcgagaagctc

SEQ ID NO: 8 - protein, 141 aa, synthetic construct
  SIGNAL 1..18
MFAKLSLLSLLFSSAALGEQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 9 - DNA, 432 bp, synthetic construct
  source 1..432: mol_type = other DNA
  sig_peptide 1..63
atgagacttccgcttgttagctcgacagtcgctcttctttccagcgccagcttagtggcagcggagcaactcaccaagtgcgaggtcttccgcgagctcaaggacctcaagggctacggcggcgtctccctccccgagtgggtctgcaccaccttccacacctccggctacgacacccaagccatcgtccaaaacaacgactccaccgagtacggcctcttccaaatcaacaacaagatctggtgcaaggacgaccaaaacccccactcctccaacatctgcaacatctcctgcgacaagttcctcgacgacgacctcaccgacgacatcatgtgcgtcaagaagatcctcgacaaggtcggcatcaactactggctcgcccacaaggccctctgctccgagaagctcgaccaatggctctgcgagaagctc

SEQ ID NO: 10 - protein, 144 aa, synthetic construct
  SIGNAL 1..21
MRLPLVSSTVALLSSASLVAAEQLTKCEVFRELKDLKGYGGVSLPEWVCTTFHTSGYDTQAIVQNNDSTEYGLFQINNKIWCKDDQNPHSSNICNISCDKFLDDDLTDDIMCVKKILDKVGINYWLAHKALCSEKLDQWLCEKL

SEQ ID NO: 11 - DNA, 1233 bp, synthetic construct
  source 1..1233: mol_type = other DNA
gaagagcagaacggaatgaagttggagcgagtcgtgattgtctccaggcacggagtccgcgcccctactaagttcacgcctatcatgaagtgcgtcacccccgaccagtggcctcagtgggacgtgcctttgggatggctcacgccgcggggatgcgagctcgtctcctacctcggccagtaccagcgcctctggttcacatcgaaaggactcttgcccaaccagacttgtccttcgcccggacaggtcgcggtcattgcggataccgaccagcgcacaaggaagaccggtgagtgcttcctcgccggtttggcgcccaaatgtcagatccaggtccattaccagaaagacgaggagaaacccgatcctttgttcaacccggtcaaaatgggcaaatgtcagttcaacactttgcaggtctgcaacgcaatcttggaacgcgcaggaggtaacattgagctcttcacacagcgataccagtcgtcgttcaggaccctcgaaaacgtcttgaacttctcgcagtcggaatgctgtaagacgaccgagaagtcgactaaatgtaccctcccggaggcattgccttccgagttgaagtgcactcccgatctcgtgtcgctcaccggcgcgtggtcgttgtcgtcgacattgacggagatcttcctcctccaggaggcccagggcatgccccaggtcgcgtggggtaggatcaccggcgagaaggagtggagggacctcttgtccttgcataacgcacagttcgacttgttgcagcgcacccccgaagtggcaaggtcgagggcaactcccttgctcgatctcatcgatactgccctcttgaccaacggcaccaccgaaaaccggtacggtatcaaattgcccgtgtccctcttgttcatcgccggccacgataccaacttggcaaacctctccggcgccctcgatctcaactggtccctccctggtcagccggataacaccccgcctggcggagagctcgtcttcgagaaatggaagcggacgtcggataacacggactgggtccaggtctccttcgtctatcagaccttgagggatatgcgtgacatccagcccctctcgctggagaagcccgccggtaaggtggacttgaaactcatcgcctgtgaggaaaagaactcgcagggtatgtgttcgctcaagtccttctcgcggctcattaaggagatccgtgtgcccgagtgtgccgtcacagag

SEQ ID NO: 12 - protein, 411 aa, synthetic construct
EEQNGMKLERVVIVSRHGVRAPTKFTPIMKCVTPDQWPQWDVPLGWLTPRGCELVSYLGQYQRLWFTSKGLLPNQTCPSPGQVAVIADTDQRTRKTGECFLAGLAPKCQIQVHYQKDEEKPDPLFNPVKMGKCQFNTLQVCNAILERAGGNIELFTQRYQSSFRTLENVLNFSQSECCKTTEKSTKCTLPEALPSELKCTPDLVSLTGAWSLSSTLTEIFLLQEAQGMPQVAWGRITGEKEWRDLLSLHNAQFDLLQRTPEVARSRATPLLDLIDTALLTNGTTENRYGIKLPVSLLFIAGHDTNLANLSGALDLNWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 13 - DNA, 1287 bp, synthetic construct
  source 1..1287: mol_type = other DNA
  sig_peptide 1..54
atgttcgccaaattgtccctcctttcgcttcttttcagctctgctgcgctgggcgaagagcagaacggaatgaagttggagcgagtcgtgattgtctccaggcacggagtccgcgcccctactaagttcacgcctatcatgaagtgcgtcacccccgaccagtggcctcagtgggacgtgcctttgggatggctcacgccgcggggatgcgagctcgtctcctacctcggccagtaccagcgcctctggttcacatcgaaaggactcttgcccaaccagacttgtccttcgcccggacaggtcgcggtcattgcggataccgaccagcgcacaaggaagaccggtgagtgcttcctcgccggtttggcgcccaaatgtcagatccaggtccattaccagaaagacgaggagaaacccgatcctttgttcaacccggtcaaaatgggcaaatgtcagttcaacactttgcaggtctgcaacgcaatcttggaacgcgcaggaggtaacattgagctcttcacacagcgataccagtcgtcgttcaggaccctcgaaaacgtcttgaacttctcgcagtcggaatgctgtaagacgaccgagaagtcgactaaatgtaccctcccggaggcattgccttccgagttgaagtgcactcccgatctcgtgtcgctcaccggcgcgtggtcgttgtcgtcgacattgacggagatcttcctcctccaggaggcccagggcatgccccaggtcgcgtggggtaggatcaccggcgagaaggagtggagggacctcttgtccttgcataacgcacagttcgacttgttgcagcgcacccccgaagtggcaaggtcgagggcaactcccttgctcgatctcatcgatactgccctcttgaccaacggcaccaccgaaaaccggtacggtatcaaattgcccgtgtccctcttgttcatcgccggccacgataccaacttggcaaacctctccggcgccctcgatctcaactggtccctccctggtcagccggataacaccccgcctggcggagagctcgtcttcgagaaatggaagcggacgtcggataacacggactgggtccaggtctccttcgtctatcagaccttgagggatatgcgtgacatccagcccctctcgctggagaagcccgccggtaaggtggacttgaaactcatcgcctgtgaggaaaagaactcgcagggtatgtgttcgctcaagtccttctcgcggctcattaaggagatccgtgtgcccgagtgtgccgtcacagag

SEQ ID NO: 14 - protein, 429 aa, synthetic construct
  SIGNAL 1..18
MFAKLSLLSLLFSSAALGEEQNGMKLERVVIVSRHGVRAPTKFTPIMKCVTPDQWPQWDVPLGWLTPRGCELVSYLGQYQRLWFTSKGLLPNQTCPSPGQVAVIADTDQRTRKTGECFLAGLAPKCQIQVHYQKDEEKPDPLFNPVKMGKCQFNTLQVCNAILERAGGNIELFTQRYQSSFRTLENVLNFSQSECCKTTEKSTKCTLPEALPSELKCTPDLVSLTGAWSLSSTLTEIFLLQEAQGMPQVAWGRITGEKEWRDLLSLHNAQFDLLQRTPEVARSRATPLLDLIDTALLTNGTTENRYGIKLPVSLLFIAGHDTNLANLSGALDLNWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 15 - DNA, 1296 bp, synthetic construct
  source 1..1296: mol_type = other DNA
  sig_peptide 1..63
atgagacttccgcttgttagctcgacagtcgctcttctttccagcgccagcttagtggcagcggaagagcagaacggaatgaagttggagcgagtcgtgattgtctccaggcacggagtccgcgcccctactaagttcacgcctatcatgaagtgcgtcacccccgaccagtggcctcagtgggacgtgcctttgggatggctcacgccgcggggatgcgagctcgtctcctacctcggccagtaccagcgcctctggttcacatcgaaaggactcttgcccaaccagacttgtccttcgcccggacaggtcgcggtcattgcggataccgaccagcgcacaaggaagaccggtgagtgcttcctcgccggtttggcgcccaaatgtcagatccaggtccattaccagaaagacgaggagaaacccgatcctttgttcaacccggtcaaaatgggcaaatgtcagttcaacactttgcaggtctgcaacgcaatcttggaacgcgcaggaggtaacattgagctcttcacacagcgataccagtcgtcgttcaggaccctcgaaaacgtcttgaacttctcgcagtcggaatgctgtaagacgaccgagaagtcgactaaatgtaccctcccggaggcattgccttccgagttgaagtgcactcccgatctcgtgtcgctcaccggcgcgtggtcgttgtcgtcgacattgacggagatcttcctcctccaggaggcccagggcatgccccaggtcgcgtggggtaggatcaccggcgagaaggagtggagggacctcttgtccttgcataacgcacagttcgacttgttgcagcgcacccccgaagtggcaaggtcgagggcaactcccttgctcgatctcatcgatactgccctcttgaccaacggcaccaccgaaaaccggtacggtatcaaattgcccgtgtccctcttgttcatcgccggccacgataccaacttggcaaacctctccggcgccctcgatctcaactggtccctccctggtcagccggataacaccccgcctggcggagagctcgtcttcgagaaatggaagcggacgtcggataacacggactgggtccaggtctccttcgtctatcagaccttgagggatatgcgtgacatccagcccctctcgctggagaagcccgccggtaaggtggacttgaaactcatcgcctgtgaggaaaagaactcgcagggtatgtgttcgctcaagtccttctcgcggctcattaaggagatccgtgtgcccgagtgtgccgtcacagag

SEQ ID NO: 16 - protein, 432 aa, synthetic construct
  SIGNAL 1..21
MRLPLVSSTVALLSSASLVAAEEQNGMKLERVVIVSRHGVRAPTKFTPIMKCVTPDQWPQWDVPLGWLTPRGCELVSYLGQYQRLWFTSKGLLPNQTCPSPGQVAVIADTDQRTRKTGECFLAGLAPKCQIQVHYQKDEEKPDPLFNPVKMGKCQFNTLQVCNAILERAGGNIELFTQRYQSSFRTLENVLNFSQSECCKTTEKSTKCTLPEALPSELKCTPDLVSLTGAWSLSSTLTEIFLLQEAQGMPQVAWGRITGEKEWRDLLSLHNAQFDLLQRTPEVARSRATPLLDLIDTALLTNGTTENRYGIKLPVSLLFIAGHDTNLANLSGALDLNWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 17 - DNA, 46 bp, Acidomyces acidothermus
  source 1..46: mol_type = genomic DNA
gtaagtaacatccactctgttctagtgccatgctgagattgtacag

SEQ ID NO: 18 - DNA, 478 bp, synthetic construct
  source 1..478: mol_type = other DNA
  sig_peptide 1..63
  intron 64..109
atgagacttccgcttgttagctcgacagtcgctcttctttccagcgccagcttagtggcagcggtaagtaacatccactctgttctagtgccatgctgagattgtacaggagcaactcaccaagtgcgaggtcttccgcgagctcaaggacctcaagggctacggcggcgtctccctccccgagtgggtctgcaccaccttccacacctccggctacgacacccaagccatcgtccaaaacaacgactccaccgagtacggcctcttccaaatcaacaacaagatctggtgcaaggacgaccaaaacccccactcctccaacatctgcaacatctcctgcgacaagttcctcgacgacgacctcaccgacgacatcatgtgcgtcaagaagatcctcgacaaggtcggcatcaactactggctcgcccacaaggccctctgctccgagaagctcgaccaatggctctgcgagaagctc

SEQ ID NO: 19 - DNA, 469 bp, synthetic construct
  source 1..469: mol_type = other DNA
  sig_peptide 1..54
  intron 55..100
atgttcgccaaattgtccctcctttcgcttcttttcagctctgctgcgctgggcgtaagtaacatccactctgttctagtgccatgctgagattgtacaggagcaactcaccaagtgcgaggtcttccgcgagctcaaggacctcaagggctacggcggcgtctccctccccgagtgggtctgcaccaccttccacacctccggctacgacacccaagccatcgtccaaaacaacgactccaccgagtacggcctcttccaaatcaacaacaagatctggtgcaaggacgaccaaaacccccactcctccaacatctgcaacatctcctgcgacaagttcctcgacgacgacctcaccgacgacatcatgtgcgtcaagaagatcctcgacaaggtcggcatcaactactggctcgcccacaaggccctctgctccgagaagctcgaccaatggctctgcgagaagctc

SEQ ID NO: 20 - DNA, 1508 bp, Aspergillus luchuensis
  source 1..1508: mol_type = genomic DNA
atgttcgccaaattgtccctcctttcgcttcttttcagctctgctgcgctgggcgcctccaaccagactctgtcctatggcaacattgacaagtctgccacccccggcgccagagcgctcctgaagtacatccagcttcagtatggatcgcactatatatccggacagcaggacgtcgacagctacaactgggtcgagaagaacattggtgtggcccctgccatcctcggcagcgacttcacctactactcgccttcggctgttgcccacggcggcaagtctcacgcggtcgaggatgtaatccagcacgccggtcgcaatggaatcaatgccctggtttggcattggtacgctcccacctgtctgctcgataccgagaaagagccgtggtacaagggattctacaccgaggccacctgcttcaacgtgtctgaagccgtcaacgaccatggcaacggcaccaactacaagctcctgctgcgtgatatcgacgccattgctgctcagatcaagcgcctggatcaggccaacgtgcccattctcttccgcccgctccacgagcccgagggtggctggttctggtggggtgcccagggtcctgctcccttcaagaagctgtgggatatcctctacgaccgcatcactcgctaccacaacctccacaacttggtctgggtttgcaacactgctgatccggcctggtatcccggaaacgacaaatgcgacatcgccaccatcgatcactatcccgccgttggtgaccacggagtcgcggccgaccagtacaagaagctccagaccctgaccaagaacgagagagttttggctatggcagaagttggccccattccggaccccgatatgcaggctcgtgagaatgtcaactgggcttactggatggtttggtccggtgagttcattgaggatggtaagcagaaccctaaccagtttttgcacaaggtgtacaacgatacccgggttgtggctctgaactgggagggtgcttaagcggacgcggtagattaccatggggggagtctaattctgagacgtgttgagcaagtgggagagaggaaatttgtggatattactaggctactccggaagtggccatcctggcagtcaacagtcactcgaagcgcctccttagctagtgtgtactattgccatacagaaataaacagtgtattacattggatcttttctcgtgtaattgattgtactaaccatccattcccgaagacctaaggggccaactgtgccaataaagtggccaagcactgatatccgctgtgggccagaaagactgaaagacggcttgaacgtcttcttcctttttcatggcgaatatgccacccacttcctgtaccgggatcaatgtgctatcttctccatctgccaggtcgcttaagtcacgccttggcttaacacacttctgggctcgtggccctgtcagaagatctggctcaactacccacgtcggggacatctggtctagaaagccttcc

SEQ ID NO: 21 - protein, 345 aa, Aspergillus luchuensis
MFAKLSLLSLLFSSAALGASNQTLSYGNIDKSATPGARALLKYIQFTLQYGSHYISGQQDVDSYNWVEKNIGVAPAILGSDFTYYSPSAVAHGGKSHAVEDVIQHAFTGRNGINALVWHWYAPTCLLDTEKEPWYKGFYTEATCFNVSEAVNDHGNGTNYKLLLRDIFTDAIAAQIKRLDQANVPILFRPLHEPEGGWFWWGAQGPAPFKKLWDILYDRITRYHNLHNFTLVWVCNTADPAWYPGNDKCDIATIDHYPAVGDHGVAADQYKKLQTLTKNERVLAMAEVGFTPIPDPDMQARENVNWAYWMVWSGEFIEDGKQNPNQFLHKVYNDTRVVALNWEGA

SEQ ID NO: 22 - DNA, 1792 bp, Aspergillus luchuensis
  source 1..1792: mol_type = genomic DNA
atgagacttccgcttgttagctcgacagtcgctcttctttccagcgccagcttagtggcagcgcagacatacaccaattgtaacccactgaagaaatgtaagtgcccaaaatcgtatgtttgatgggcaggcgcagctaatacttctaaagcatgtcctgcggatcccgcactgggcaaatctgcgagctatgacttcactcaaggcagttcgtccgactttactgcaaccggtagcccgacctatggtgacaatggtgccgagttcaccgtagctcaacagggagatgcgccattgatccagtccaactggtacatcatgtttgggcacgtggaatacgtgatcaaggctgcccctggaacgggcattgtcagcagtgccgttttgcagtccgatgacctggatgagatcgactgggaatggcttggagggaacgaccagcttgtgcaaaccaactacttcggaaaaggagatacaagtagctacaatcgtggagctacccataatgatcctgggaaccacgacgactttcacacctatactgtggactggacgagcaagcaaatcgtctggcagatcgatggcacaacggtcagagtgcttacgccggatactgccgacacgaaccagtacccccaaacccccatggttgtcaaagtaggagtctgggctggaggcgatcccagtaaccctcaaggtacaatcggtgagtgtctagccacatgtccctgcagtgcttgccactggtttgcaatatgctaatgtttgtcttagattgggctggaggcacgaccgattacagcgccggaccctacacaatgtacttgaagtccctcaaagtgacggactattcgactggctcgtcatactcatatggtgatgagagtggggactggacgtcaatcactgccaacggaggccaggttaatggaaacattggggccgatacaatgacctcaattgagtcagcacccactgtgacgtcgacagttgccagtatccctatcccttggagcggcactcatcgtgaaacatccagctttgtgactccagatgtctggccttgggttgcgacagcaacatcgacatcgctgccttctggctggacatattctggttctagacaaatccagccgcctagcgcggcttctgtgagtgagcatttcccttaacacccttttatacaaacctcaggatctcacgctaaatcggtactgcttttagtcttcctcccagtgtatatcagcctcctcgcaatctcggccggcttcattctccccctccgtctctaaatcccgctcaagaactccaagtttgactgcttcctcgacgactgctgctggacaaactagttccgccactcatgcgggtagctccaccacgaccagacatctctctcctacttcaaccaatcacgcagccccaaatgtgaatgctgcgggaagaccaaataggattcacattggcatgcatgtgatatgtgtgcttattagcacagtgatcatgctctgatacgctgcgcaacgaccgttgaaaaattgatcaggtctatacattaccagcaccgctactcgacgacagttatgacagttatgcatacgacacgacgactactataacacgacatcacgacgaagtgtatatgagtgattggcccctttccttcttcctcgtttatgatagagatcttcatgagctatgtacagcatagggttggcgttgaatcacgaaagaggcgagtcagacgagctcgctaattgcaccacgtgctgtggctttattggatcaattg

SEQ ID NO: 23 - protein, 366 aa, Aspergillus luchuensis
MRLPLVSSTVALLSSASLVAAQTYTNCNPLKKSCPADPALGKSASYDFTQGSSSDFTATGSPTYGDNGAEFTVAQQGDAPLIQSNWYIMFGHVEYVIKAAPGTGIVSSAVLQSDDLDEIDWEWLGGNDQLVQTNYFGKGDTSSYNRGATHNDPGNHDDFHTYTVDWTSKQIVWQIDGTTVRVLTPDTADTNQYPQTPMVVKVGVWAGGDPSNPQGTIDWAGGTTDYSAGPYTMYLKSLKVTDYSTGSSYSYGDESGDWTSITANGGQVNGNIGADTMTSIESAPTVTSTVASIPIPWSGTHRETSSFVTPDVWPWVATATSTSLPSGWTYSGSRQIQPPSAASVIFLPVYISLLAISAGFILPLRL

SEQ ID NO: 24 - DNA, 72 bp, Aspergillus niger
  source 1..72: mol_type = genomic DNA
atgactccaactatggtcgcgtggtggtctctatttctgtacggccttcaggtcgcggcacctgctttggct

SEQ ID NO: 25 - protein, 23 aa, Aspergillus niger
MTPMVAWWSLFLYGLQVAAPALA

SEQ ID NO: 26 - DNA, 105 bp, Humicola insolens
  source 1..105: mol_type = genomic DNA
atgaagttcttcaccacgatcctctcgactgcctcgttggtcgcagccttgcctgcggcagtggattccaaccacacaccggcagcacccgagctcgtggcacgc

SEQ ID NO: 27 - protein, 35 aa, Humicola insolens
MKFFTTILSTASLVAALPAAVDSNHTPAAPELVAR

SEQ ID NO: 28 - DNA, 60 bp, Aspergillus niger
  source 1..60: mol_type = genomic DNA
atgaagttctccgctactacccttgctggagcttcgctcctcctgggccgcgccattgcc

SEQ ID NO: 29 - protein, 20 aa, Aspergillus niger
MKFSATTLAGASLLLGRAIA

SEQ ID NO: 30 - DNA, 2112 bp, synthetic construct
  source 1..2112: mol_type = other DNA
ccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagcaacaatctcagaacaccaatatcaattcatctcgagtctgaactcagacaacacaaattcttcaaaattgaggatttagtcttgatcggatccaccatgagacttccgcttgttagctcgacagtcgctcttctttccagcgccagcttagtggcagcggtaagtaacatccactctgttctagtgccatgctgagattgtacaggagcaactcaccaagtgcgaggtcttccgcgagctcaaggacctcaagggctacggcggcgtctccctccccgagtgggtctgcaccaccttccacacctccggctacgacacccaagccatcgtccaaaacaacgactccaccgagtacggcctcttccaaatcaacaacaagatctggtgcaaggacgaccaaaacccccactcctccaacatctgcaacatctcctgcgacaagttcctcgacgacgacctcaccgacgacatcatgtgcgtcaagaagatcctcgacaaggtcggcatcaactactggctcgcccacaaggccctctgctccgagaagctcgaccaatggctctgcgagaagctctagcacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccagatccgggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccgatcattgatccaccgcccacgaggcgcgtctttgctttttgcgcggcgtccaggttcaactctctcc

SEQ ID NO: 31 - DNA, 2103 bp, synthetic construct
  source 1..2103: mol_type = other DNA
ccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagcaacaatctcagaacaccaatatcaattcatctcgagtctgaactcagacaacacaaattcttcaaaattgaggatttagtcttgatcggatccaccatgttcgccaaattgtccctcctttcgcttcttttcagctctgctgcgctgggcgtaagtaacatccactctgttctagtgccatgctgagattgtacaggagcaactcaccaagtgcgaggtcttccgcgagctcaaggacctcaagggctacggcggcgtctccctccccgagtgggtctgcaccaccttccacacctccggctacgacacccaagccatcgtccaaaacaacgactccaccgagtacggcctcttccaaatcaacaacaagatctggtgcaaggacgaccaaaacccccactcctccaacatctgcaacatctcctgcgacaagttcctcgacgacgacctcaccgacgacatcatgtgcgtcaagaagatcctcgacaaggtcggcatcaactactggctcgcccacaaggccctctgctccgagaagctcgaccaatggctctgcgagaagctctagcacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccagatccgggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccgatcattgatccaccgcccacgaggcgcgtctttgctttttgcgcggcgtccaggttcaactctctcc

SEQ ID NO: 32 - DNA, 2109 bp, synthetic construct
  source 1..2109: mol_type = other DNA
ccgttaaattgccgtcgtcagccgttaaattaccgattaatcccgataaatttccgagatctccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagcaacaatctcagaacaccaatatcaattcatctcgagtctgaactcagacaacacaaattcttcaaaattgaggatttagtcttgatcggatccaccatgaagttctccgctactacccttgctggagcttcgctcctcctgggccgcgccattgccgtaagtaacatccactctgttctagtgccatgctgagattgtacaggagcaactcaccaagtgcgaggtcttccgcgagctcaaggacctcaagggctacggcggcgtctccctccccgagtgggtctgcaccaccttccacacctccggctacgacacccaagccatcgtccaaaacaacgactccaccgagtacggcctcttccaaatcaacaacaagatctggtgcaaggacgaccaaaacccccactcctccaacatctgcaacatctcctgcgacaagttcctcgacgacgacctcaccgacgacatcatgtgcgtcaagaagatcctcgacaaggtcggcatcaactactggctcgcccacaaggccctctgctccgagaagctcgaccaatggctctgcgagaagctctagcacgtggtgcgtacgataacgcatagtgtttttccctccacttaaatcgaagggttgtgtcttggatcgcgcgggtcaaatgtatatggttcatatacatccgcaggcacgtaataaagcgaggggttcgaatccccccgttacccccggtaggggcccagatccgggtgactgacacctggcggtagacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccgatcattgatccaccgcccacgaggcgcgtctttgctttttgcgcggcgtccaggttcaactctctcc

SEQ ID NO: 33 - DNA, 2683 bp, synthetic construct
  source 1..2683: mol_type = other DNA
ccgttaaattgccgttcgcagccgttaaattaccggggacgaccgataaatttccgcgatgaattcatggtgttttgatcattttaaatttttatatggcgggtggtgggcaactcgcttgcgcgggcaactcgcttaccgattacgttagggctgatatttacgtaaaaatcgtcaagggatgcaagaccaaaccgttaaatttccggagtcaacagcatccaagcccaagtccttcacggagaaaccccagcgtccacatcacgagcgaaggaccacctctaggcatcggacgcaccatccaattagaagcagcaaagcgaaacagcccaagaaaaaggtcggcccgtcggccttttctgcaacgctgatcacgggcagcgatccaaccaacaccctccagagtgactaggggcggaaatttatcgggattaatttccactcaaccacaaatcacagtcgtccccggtaatttaacggctgcagacggcaatttaacggcttctgcgaatcgcttggattccccgcccctggccgtagagcttaaagtatgtcccttgtcgatgcgatgtatcacaacatataaatactggcaagggatgccatgcttggagcaacaatctcagaacaccaatatcaattcatctcgagtctgaactcagacaacacaaattcttcaaaattgaggatttagtcttgatcggatccatgagacttccgcttgttagctcgacagtcgctcttctttccagcgccagcttagtggcagcggaagagcagaacggaatgaagttggagcgagtcgtgattgtctccaggcacggagtccgcgcccctactaagttcacgcctatcatgaagtgcgtcacccccgaccagtggcctcagtgggacgtgcctttgggatggctcacgccgcggggatgcgagctcgtctcctacctcggccagtaccagcgcctctggttcacatcgaaaggactcttgcccaaccagacttgtccttcgcccggacaggtcgcggtcattgcggataccgaccagcgcacaaggaagaccggtgagtgcttcctcgccggtttggcgcccaaatgtcagatccaggtccattaccagaaagacgaggagaaacccgatcctttgttcaacccggtcaaaatgggcaaatgtcagttcaacactttgcaggtctgcaacgcaatcttggaacgcgcaggaggtaacattgagctcttcacacagcgataccagtcgtcgttcaggaccctcgaaaacgtcttgaacttctcgcagtcggaatgctgtaagacgaccgagaagtcgactaaatgtaccctcccggaggcattgccttccgagttgaagtgcactcccgatctcgtgtcgctcaccggcgcgtggtcgttgtcgtcgacattgacggagatcttcctcctccaggaggcccagggcatgccccaggtcgcgtggggtaggatcaccggcgagaaggagtggagggacctcttgtccttgcataacgcacagttcgacttgttgcagcgcacccccgaagtggcaaggtcgagggcaactcccttgctcgatctcatcgatactgccctcttgaccaacggcaccaccgaaaaccggtacggtatcaaattgcccgtgtccctcttgttcatcgccggccacgataccaacttggcaaacctctccggcgccctcgatctcaactggtccctccctggtcagccggataacaccccgcctggcggagagctcgtcttcgagaaatggaagcggacgtcggataacacggactgggtccaggtctccttcgtctatcagaccttgagggatatgcgtgacatccagcccctctcgctggagaagcccgccggtaaggtggacttgaaactcatcgcctgtgaggaaaagaactcgcagggtatgtgttcgctcaagtccttctcgcggctcattaaggagatccgtgtgcccgagtgtgccgtcacagagtagcacgtgacaatcaatccatttcgctatagttaaaggatggggatgagggcaattggttatatgatcatgtatgtagtgggtgtgcataatagtagtgaaatggaagccaagtcatgtgattgtaatcgaccgacggaattgaggatatccggaaatacagacaccgtgaaagccatggtctttccttcgtgtagaagaccagacagacagtccctgatttacccttgcacaaagcactagaaaattagcattccatccttctctgcttgctctgctgatatcactgtcattcaatgcatagccatgagctcatcttagatccaagcacgtaattccatagccgaggtccacagtggagcagcaacattccccatcattgctttccccaggggcctcccaacgactaaatcaagagtatatctctaccgtccaatagatcgtcttcgcttcaaaatctttgacaattccaagagggtccccatccatcaaacccagttcaataatagccgagatgcatggtggagtcaattaggcagtattgctggaatgtcggggccagttggcccggtggtcattggccgcctgtgatgccatctgccactaaatccgatcattgatccaccgcccacgaggcgcgtctttgctttttgcgcggcgtccaggttcaactctctcc

SEQ ID NO: 34 - DNA, 21 bp, synthetic construct
  source 1..21: mol_type = other DNA
gaagagcagaacggaatgaag

SEQ ID NO: 35 - DNA, 20 bp, synthetic construct
  source 1..20: mol_type = other DNA
cttccttgaactctcaagcc

SEQ ID NO: 36 - DNA, 20 bp, synthetic construct
  source 1..20: mol_type = other DNA
cttccttgaactctcaagcc

SEQ ID NO: 37 - DNA, 32 bp, synthetic construct
  source 1..32: mol_type = other DNA
ccgttctgctcttccgctgccactaagctggc

SEQ ID NO: 38 - DNA, 26 bp, synthetic construct
  source 1..26: mol_type = other DNA
ccgttctgctcttcgcccagcgcagc

SEQ ID NO: 39 - DNA, 29 bp, synthetic construct
  source 1..29: mol_type = other DNA
ccgttctgctcttcagccaaagcaggtgc

SEQ ID NO: 40 - DNA, 21 bp, synthetic construct
  source 1..21: mol_type = other DNA
gaagagcagaacggaatgaag

SEQ ID NO: 41 - DNA, 22 bp, synthetic construct
  source 1..22: mol_type = other DNA
cattacttcgcactagtctccc

SEQ ID NO: 42 - DNA, 22 bp, synthetic construct
  source 1..22: mol_type = other DNA
gggagactagtgcgaagtaatg

SEQ ID NO: 43 - DNA, 106 bp, synthetic construct
  source 1..106: mol_type = other DNA
cttcattccgttctgctcttcggcaatggcgcggcccaggaggagcgaagctccagcaagggtagtagcggagaacttcatggtggatccgatcaagactaaatcc

SEQ ID NO: 44 - DNA, 57 bp, Aspergillus luchuensis
  source 1..57: mol_type = genomic DNA
atgaagtacgctgctgctcttacggctgttgccgccctcgctgcccgcgccgctgct

SEQ ID NO: 45 - protein, 19 aa, Aspergillus luchuensis
MKYAAALTAVAALAARAAA

SEQ ID NO: 46 - DNA, 60 bp, Aspergillus niger
  source 1..60: mol_type = genomic DNA
atggtcgtcttcagcaaaaccgctgccctcgttctgggtctgtccaccgccgtctctgcg

SEQ ID NO: 47 - protein, 20 aa, Aspergillus niger
MVVFSKTAALVLGLSSAVSA

SEQ ID NO: 48 - DNA, 60 bp, Aspergillus luchuensis
  source 1..60: mol_type = genomic DNA
atgcatttcctccagaacgctttcgtggccgccactatgggcgctgcgctggccgccgcc

SEQ ID NO: 49 - protein, 20 aa, Aspergillus luchuensis
MHFLQNAFVAATMGAALAAA